(19) United States
(12) Patent Application Publication
(10) Pub. No.: US 2026/0007898 A1
(43) Pub. Date: Jan. 8, 2026

(54) Body Maps for Spinal Cord Stimulation Targeting

(71) Applicant: Boston Scientific Neuromodulation Corporation, Valencia, CA (US)

(72) Inventors: Bradley Hershey, Carrollton, TX (US); Tianhe Zhang, Studio City, CA (US); Lisa Moore, Glendale, CA (US); Marcus Harvey, San Francisco, CA (US); Amarpreet Bains, Woodbury, MN (US); Diana DeCarlo, Roseville, CA (US); Jianwen Gu, Richmond, TX (US); G. Karl Steinke, Valencia, CA (US); Que Doan, West Hills, CA (US)

(21) Appl. No.: 19/256,892

(22) Filed: Jul. 1, 2025

Related U.S. Application Data
(60) Provisional application No. 63/668,093, filed on Jul. 5, 2024.
(60) Provisional application No. 63/751,682, filed on Jan. 30, 2025.

(51) Int. Cl. A61N 1/372; A61N 1/36

(52) U.S. Cl. CPC A61N 1/37247; A61N 1/36071; A61N 1/36185; A61N 1/37241

(57) ABSTRACT

Methods and systems for determining an optimal stimulation location in an electrode array of a neurostimulator are disclosed. An external system such as a clinician programmer includes a targeting algorithm implemented as part of a graphical user interface. The disclosure describes a user interface providing body maps for inputting pain/paresthesia locations based on intuitive body locations. Information from the body maps may be translated to a physiological or anatomical reference frame to provide information that can be used by the targeting algorithm for determining stimulation parameters that are likely to be therapeutic for the patient.

### CROSS REFERENCE TO RELATED APPLICATIONS

This is a non-provisional application of U.S. Provisional Patent Application Ser. Nos. 63/751,682, filed Jan. 30, 2025, and 63/668,093, filed Jul. 5, 2024, which are incorporated by reference in their entirety, and to which priority is claimed.

### FIELD OF THE INVENTION

This application relates to Implantable Medical Devices (IMDs) generally, Spinal Cord Stimulators more specifically, and to methods of control and programming of such devices.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible device case 12 that holds the circuitry and battery 14 necessary for the IPG to function. The IPG 10 is coupled to electrodes 16 via one or more percutaneous electrode leads 15 that form an electrode array 17. The electrodes 16 are configured to contact a patient's tissue and are carried on a flexible body 18, which also houses the individual lead wires 20 coupled to each electrode 16. The lead wires 20 are also coupled to proximal contacts 22, which are insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins through a case feedthrough to stimulation circuitry 28 within the case 12, although these details aren't shown. The electrode array 17 can also be provided on a paddle lead 19 comprising a matrix of electrodes 16.

In the illustrated IPG 10, there are sixteen lead electrodes (E1-E16) split between two leads 15, with the header 23 containing a 2×1 array of lead connectors 24. However, the number of leads and electrodes in an IPG is application specific and therefore can vary. The conductive case 12 can also comprise an electrode (Ec). In a SCS application, the electrode array 17 (leads 15 or paddle 19) are typically implanted proximate to the dura in a patient's spinal column such that the electrodes are located on the right and left sides of the spinal cord midline. The proximal electrodes 22 are tunneled through the patient's tissue to a distant location such as the buttocks where the IPG case 12 is implanted, at which point they are coupled to the lead connectors 24. In other IPG examples designed for implantation directly at a site requiring stimulation, the IPG can be lead-less, having electrodes 16 instead appearing on the body of the IPG for contacting the patient's tissue. The electrode array 17 can be integrated with and permanently connected the case 12 in other IPG solutions. The goal of SCS therapy is to provide electrical stimulation from the electrodes 16 to alleviate a patient's symptoms, most notably chronic back pain.

Although not shown, IPG 10 is also representative of an External Trial Stimulator (ETS), which is a device that is functionally similar to the IPG 10 but whose use precedes implantation of the IPG 10 in a patient. During external trial stimulation, stimulation can be tried on a prospective implant patient without going so far as to implant the IPG 10. One or more trial leads are implanted as an electrode array 17 in the spinal column as explained earlier. The proximal ends of the (transcutaneous) trial lead(s) exit an incision and are connected to an ETS. The ETS generally mimics operation of the IPG 10, and thus can provide stimulation to the patient's tissue. See, e.g., 9,259,574, disclosing a design for an ETS. The ETS is worn externally by the patient for a short while (e.g., two weeks), which allows the patient and his clinician to experiment with different stimulation parameters to try and find a stimulation program that alleviates the patient's symptoms (e.g., pain). If external trial stimulation proves successful, trial lead(s) are explanted, and a permanent IPG 10 and electrode array 17 are implanted as described above; if unsuccessful, the trial lead(s) are simply explanted. IPG 10 as described herein should be understood as including stimulator devices such as ETSs.

IPG 10 can include an antenna 27a allowing it to communicate bi-directionally with a number of external devices, as explained later with respect to FIG. 4. The antenna 27a as depicted in FIG. 1 is shown as a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. In FIG. 1, RF antenna 27b is shown within the header 23, but it may also be within the case 12. RF antenna 27b may comprise a patch, slot, or wire, and may operate as a monopole or dipole. RF antenna 27b preferably communicates using far-field electromagnetic wave, and may operate in accordance with any number of known RF communication standards, such as Bluetooth, Zigbee, WiFi, MICS, and the like.

Stimulation in IPG 10 is typically provided by pulses, as shown in FIG. 2. Stimulation parameters typically include the amplitude of the pulses (I; whether current or voltage); the frequency (F) and pulse width (PW) of the pulses; the electrodes 16 (E) activated to provide such stimulation; and the polarity of such active electrodes, i.e., whether active electrodes are to act as anodes (that source current to the tissue) or cathodes (that sink current from the tissue). These stimulation parameters taken together comprise a stimulation program that the IPG 10 can execute to provide therapeutic stimulation to a patient.

In the example of FIG. 2, electrode E1 has been selected as an anode (during first phase 30a), and thus provides pulses which source a positive current of amplitude +I to the tissue. Electrode E2 has been selected as a cathode (during first phase 30a), and thus provides pulses which sink a corresponding negative current of amplitude −I from the tissue. This respectively provides an anode pole (+) and a cathode pole (−) at these electrodes, and provides an example of bipolar stimulation, in which these two poles in the electrode array 17 are used to provide stimulation to the tissue. The resulting bipole 21 can be said to have a particular location L in the electrode array 17, which may be differently defined with respect to the anode and cathode. In the example shown this location L is defined as the midway position between the two poles. In another example, the location of the bipole 21 can be defined with respect to only one of the poles, such as the cathode. As explained further below, more than one anode electrode can be active at a given time to establish an anode pole, and more than one cathode electrode may be active at a given time to establish a cathode pole. This allows the location of the anode and cathode poles to be moved or steered in the electrode array in X and Y direction, again as discussed further below.

The pulses as shown in FIG. 2 are biphasic, comprising a first phase 30a, followed quickly thereafter by a second phase 30b of opposite polarity. As is known, use of a biphasic pulse is useful in active charge recovery. For example, each electrodes' current path to the tissue may include a serially-connected DC-blocking capacitor 38, see FIG. 3, which will charge during the first phase 30a and be discharged (charge will be actively recovered) during the second phase 30b. In the example shown, the first and second phases 30a and 30b have the same pulse width (PW) and amplitude (although of opposite polarities), which ensures the same amount of charge is injected and recovered at each electrode during both phases (+Q/−Q). However, the second phase 30b may also be charged balance with the first phase 30a if the integral of the amplitude (i.e., the area) of the two phases are equal in magnitude, as is well known.

IPG 10 includes stimulation circuitry 28 to form prescribed stimulation pulses, and FIG. 3 shows an example of such circuitry, which includes one or more current sources 40; and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current through a patient's tissue, Z, hopefully with good therapeutic effect. In the example shown, and consistent with the first pulse phase 30a of FIG. 2, electrode E1 has been selected as an anode electrode to source current I to the tissue Z and electrode E2 has been selected as a cathode electrode to sink current I from the tissue. Thus PDAC 40₁ and NDAC 42₂ are activated and digitally programmed to produce the desired current, I, with the correct timing (e.g., in accordance with the prescribed frequency F and pulse width PW). During the second phase 30b, the direction of the current through the tissue would be reversed by activating PDACs 40₂ and NDACs 42ᵢ to produce current I. Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40; and the electrode nodes ci 39, and between the one or more NDACs 42; and the electrode nodes. Switching matrices allow one or more of the PDACs or one or more of the NDACs to be connected to one or more electrode nodes at a given time. Various examples of stimulation circuitries can be found in U.S. Pat. Nos. 6,181,969, 8,606,362, 8,620,436, U.S. Patent Application Publications 2018/0071520 and 2019/0083796.

Much of the stimulation circuitry 28 of FIG. 3, including the PDACs 40; and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ci 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

The stimulation circuitry 28 can include passive recovery switches 41ᵢ, which are described further in U.S. Patent Application Publications 2018/0071527 and 2018/0140831. Passive recovery switches 41ᵢ may be closed to passively recover any charge remaining on the DC-blocking capacitors Ci 38 after issuance of a pulse (e.g., after second pulse phase 30b during phase 30c as shown in FIG. 2) to recover charge without actively driving a current using the DAC circuitry, as shown during duration 30c. This is beneficial, because active charge recovery using biphasic pulses as described earlier may not operate perfectly, and therefore some small amount of residual charge may still require passive recovery. Again, passive charge recovery is well known and not further described. Although not shown, the stimulation pulses may also be monophasic comprising single actively-driven phases (e.g., 30a), each followed by passive charge recovery (e.g., 30c).

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information and measurements, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with a compatible antenna 27a and/or 27b in the IPG 10, such as a near-field magnetic-induction coil antenna 64a and/or a far-field RF antenna 64b.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller. A communication “wand” 76 coupleable to suitable ports on the computing device can include an IPG-compliant antenna such as a coil antenna 74a or an RF antenna 74b. The computing device itself may also include one or more RF antennas 74b. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with IPG communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. Memory 94 may comprise solid state, optical, or magnetic memories for example capable of storing the software 96 in a non-transitory fashion. In external system 80, the external system software 96 would likely reside in the server 86, while the control circuitry 92 could be present in either or both the server 86 or the terminal 87.

### SUMMARY

Disclosed herein is an external system for programming stimulation for a spinal cord stimulator implanted in a patient to treat the patient's pain, wherein the spinal cord stimulator comprises an electrode array implantable in the patient's spinal column, the external system comprising: a user interface and control circuitry configured to: cause the user interface to present one or more body maps comprising selectable body map regions, receive indications of one or more selected body map regions corresponding to body locations where the patient feels the pain, cause stimulation circuitry of the spinal cord stimulator to provide one or more test stimulations, receive indications of one or more selected body map regions corresponding to body locations where the patient feels sensations evoked by the one or more test stimulations, and use a translation algorithm to determine mediolateral locations and dermatomes where the patient feels the pain and the sensations based on the selected body map regions. According to some embodiments, the one or more body maps do not provide any indication of dermatome boundaries. According to some embodiments, the translation algorithm is configured to determine overlaps of the selected body map regions with dermatomes in the patient's body. According to some embodiments, the translation algorithm is configured to determine a correlation between the selected body map regions with dermatomes in the patient's body. According to some embodiments, the translation algorithm is configured to score the correlation. According to some embodiments, the translation algorithm is configured to generate one or more basis functions for each of the selected body map regions, wherein each of the basis functions comprises a mediolateral index and one or more coefficients indicative of dermatomes that overlap each of the respective selected body map regions. According to some embodiments, the one or more coefficients are determined based on a number of pixels representing the selected body map regions that overlap with pixels representing respective dermatomes. According to some embodiments, the translation algorithm determines an overlap ratio based on the overlapping pixels and a total number of pixels representing the respective dermatomes. According to some embodiments, the coefficient of a respective dermatome indicates that the respective dermatome is overlapped if the overlap ratio exceeds a predefined threshold value. According to some embodiments, the mediolateral locations and dermatomes where the patient feels the pain and the sensations are each determined based on mathematical combinations of their respective basis functions. According to some embodiments, the control circuitry is configured to: use the mediolateral locations and dermatomes where the patient feels the pain and the sensations to automatically determine one or more therapeutic locations in the electrode array where stimulation is likely to treat the patient's pain, and provide stimulation at the one or more therapeutic locations. According to some embodiments, the one or more test stimulations comprise at least a first test stimulation using a first test stimulation location in the array and a second test stimulation using a second test stimulation location in the array. According to some embodiments, the control circuitry is configured to use the mediolateral locations and dermatomes where the patient feels the pain and the sensations to determine: one or more pain inputs indicative of mediolateral and rostro-caudal locations on the patient's body where the patient feels the pain, one or more first inputs indicative of mediolateral and rostro-caudal locations on the patient's body where the patient feels sensations evoked by the first test stimulation, and one or more second inputs indicative of mediolateral and rostro-caudal locations on the patient's body where the patient feels sensations evoked by the second test stimulation. According to some embodiments, the control circuitry is configured to: average the one or more pain inputs, average the one or more first inputs, average the one or more second inputs, and automatically determine the optimal location using the averaged pain inputs, the averaged first inputs, the averaged second inputs, the first test stimulation location, and the second stimulation location. According to some embodiments, the control circuitry is configured to: combine information from the one or more pain inputs, combine information from the one or more first inputs, combine information from the one or more second inputs, and automatically determine the one or more therapeutic locations using the combined pain inputs, the combined first inputs, the combined second inputs, the first test stimulation location, and the second stimulation location. According to some embodiments, determining the one or more therapeutic locations in the electrode array comprises determining a midline in the electrode array corresponding to a physiological midline of the patient. According to some embodiments, the one or more therapeutic locations are automatically determined as a location on the midline. According to some embodiments, the therapeutic location(s) are automatically determined as an offset from the midline. According to some embodiments, the offset from the midline is determined along an axis perpendicular to the midline. According to some embodiments, the first and second test stimulations are moved in the electrode array using the user interface to a first test stimulation location where the sensations evoked by the first test stimulation is felt at symmetric body locations, and wherein sensations evoked by the second test stimulation is felt at symmetric body locations. According to some embodiments, the first and second test stimulations are moved medio-laterally in the electrode array using the user interface to first and second test stimulation locations, wherein the first test stimulation once moved is felt medio-laterally symmetrically on the body at the one or more first sites, and wherein the second test stimulation once moved is felt medio-laterally symmetrically on the body at the one or more second sites. According to some embodiments, the first and second test stimulation locations are respectively located at top and bottom locations in the electrode array. According to some embodiments, the control circuitry is further configured to receive an indication of patient feedback concerning the stimulation provided at the optimal location, and use the user interface to adjust the stimulation to a further beneficial location in the electrode array to even better treat the pain at the one or more pain sites. According to some embodiments, the user interface comprises one or more selection elements configured to assist a user in selecting the selectable body map regions. According to some embodiments, the one or more selection elements comprise a cursor. According to some embodiments, the one or more selection elements comprise a pen or stylus, whereby a user can mark across multiple selectable body map regions to simultaneously select each of them. According to some embodiments, the one or more selection elements comprise a symmetry function, configured such that when a user selects a first body map region on a first half of the body map a second body map region symmetrically positioned on a second half of the body map is also selected. According to some embodiments, the one or more selection elements comprise a magnifying element, which when positioned over a portion of the body map magnifies selectable body map regions within the body map. According to some embodiments, the one or more selection elements comprise a speech-to-text functionality configured to select body map regions based on verbal input from a user. According to some embodiments, the one or more selection elements comprise a chat tool configured to present instructions to a user to aid the user's selection of body map regions. According to some embodiments, the chat tool is configured with natural language processing capabilities. According to some embodiments, the control circuitry is configured to adjust the number of selectable body map regions contained in the body maps. According to some embodiments, the control circuitry is configured to adjust coarseness of the selectable body map regions contained in the body maps.

Also disclosed herein is an external system for programming stimulation for a neurostimulator implanted in a patient, wherein the neurostimulator comprises an electrode array implantable in the patient, the external system comprising: a user interface and control circuitry configured to: cause the user interface to present one or more body maps comprising selectable body map regions, cause stimulation circuitry of the neurostimulator to provide one or more test stimulations, receive indications of one or more selected body map regions corresponding to body locations where the stimulation induces an effect, and use a translation algorithm to correlate the selected body map regions to a physiological reference frame. According to some embodiments, the one or more body maps do not provide any indication of the physiological reference frame. According to some embodiments, the physiological reference frame comprises dermatomes. According to some embodiments, the translation algorithm is configured to determine overlaps of the selected body map regions with locations within the physiological reference frame. According to some embodiments, the translation algorithm is configured to score the correlation of the selected body map regions to the physiological reference frame. According to some embodiments, the translation algorithm is configured to generate one or more basis functions for each of the selected body map regions, wherein each of the basis functions comprises one or more coefficients indicative of locations in the physiological reference frame that overlap each of the respective selected body map regions. According to some embodiments, the one or more coefficients are determined based on a number of pixels representing the selected body map regions that overlap with pixels representing respective locations in the physiological reference frame. According to some embodiments, the translation algorithm determines an overlap ratio based the overlapping pixels and a total number of pixels representing the respective locations in the physiological reference frame. According to some embodiments, the coefficient of a respective dermatome indicates that the respective location in the physiological reference frame is overlapped if the overlap ratio exceeds a predefined threshold value. According to some embodiments, the user interface comprises one or more selection elements configured to assist a user in selecting the selectable body map regions. According to some embodiments, the one or more selection elements comprise a cursor. According to some embodiments, the one or more selection elements comprise a pen or stylus, whereby a user can mark across multiple selectable body map regions to simultaneously select each of them. According to some embodiments, the one or more selection elements comprise a symmetry function, configured such that when a user selects a first body map region on a first half of the body map a second body map region symmetrically positioned on a second half of the body map is also selected. According to some embodiments, the one or more selection elements comprise a magnifying element, which when positioned over a portion of the body map magnifies selectable body map regions within the body map. According to some embodiments, the one or more selection elements comprise a speech-to-text functionality configured to select body map regions based on verbal input from a user. According to some embodiments, the one or more selection elements comprise a chat tool configured to present instructions to a user to aid the user's selection of body map regions. According to some embodiments, the control circuitry is configured to adjust the number of selectable body map regions contained in the body maps. According to some embodiments, the control circuitry is configured to adjust coarseness of the selectable body map regions contained in the body maps.

The invention may also reside in the methods performed by the above-described systems, graphical user interfaces for facilitating those methods, and non-transitory computer readable media containing instructions for programming a system and/or an external device (via its control circuitry) for carrying out the methods, a programmed implantable pulse generator (IPG) or external trial stimulator (ETS), external pulse generator (EPG), (via their respective control circuitry) for carrying out the above methods, a system including a programmed external device and IPG or ETS for carrying out the above methods, or as a computer-readable media for carrying out the above methods stored in an external device or IPG or ETS.

### BRIEF DESCRIPTION OF THE DRAWINGS

FIG. 1 shows a Spinal Cord Stimulation (SCS) Implantable Pulse Generator (IPG) and its electrode array, in accordance with the prior art.

FIG. 2 shows an example of stimulation pulses producible by the IPG, in accordance with the prior art.

FIG. 3 shows stimulation circuitry useable in an IPG, in accordance with the prior art.

FIG. 4 shows various external devices capable of communicating with and programming stimulation in an IPG, in accordance with the prior art.

FIGS. 5A and 5B show a Graphical User Interface (GUI) of a clinician programmer external system for setting or adjusting the location of simulation in the electrode array.

FIG. 6 shows a map of human dermatomes.

FIG. 7 shows a targeting algorithm operable in the clinician programmer for determining an optimal location to provide stimulation for a patient.

FIGS. 8A-8F show various interfaces for providing patient pain information to the targeting algorithm.

FIGS. 9A-9C show testing and adjustment of top and bottom test stimulation, and interfaces for providing affected dermatome information to the targeting algorithm.

FIGS. 10A-10F show details of a first example of a position algorithm in the targeting algorithm which determines an optimal location to provide stimulation for a patient using information gathered during FIGS. 8A-9C.

FIGS. 11A-11D show details of a second example of a position algorithm in which a medio-lateral offset can be automatically determined and applied.

FIGS. 12A-12D show details of a third example of a position algorithm using a physiological map.

FIGS. 13A-13E show details of a fourth example of a position algorithm which determines and uses a therapy map.

FIG. 14 shows an example of a user interface with body maps and selectable body map regions for inputting pain/paresthesia locations.

FIG. 15 shows aspects of selecting body map regions using an example of a user interface with body maps and selectable body map regions for inputting pain/paresthesia locations.

FIG. 16 shows examples of selection elements according to some embodiments of a user interface with body maps and selectable body map regions for inputting pain/paresthesia locations.

FIG. 17 shows examples of denoting selected body map regions according to some embodiments of a user interface with body maps and selectable body map regions for inputting pain/paresthesia locations.

FIG. 18 shows an embodiment of a workflow of translating from selected body map regions to mediolateral locations and vertebral levels.

FIG. 19 shows an embodiment of constructing selectable body map regions mapped to dermatomal locations.

FIG. 20 shows of basis functions relating selected body map regions to mediolateral values and dermatomal locations.

FIG. 21 shows a matrices of dermatomal coefficients and mediolateral values.

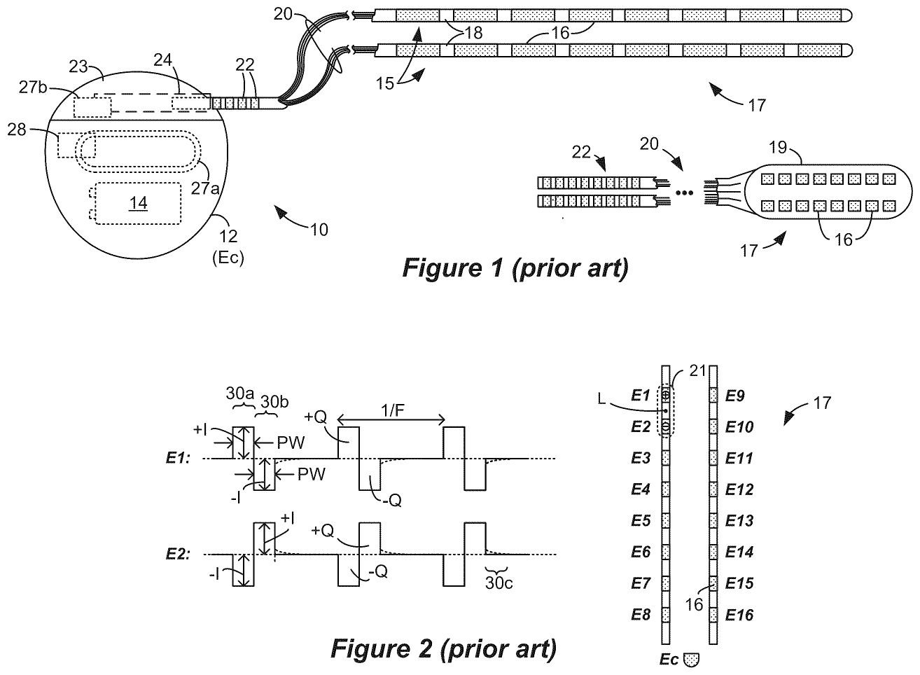

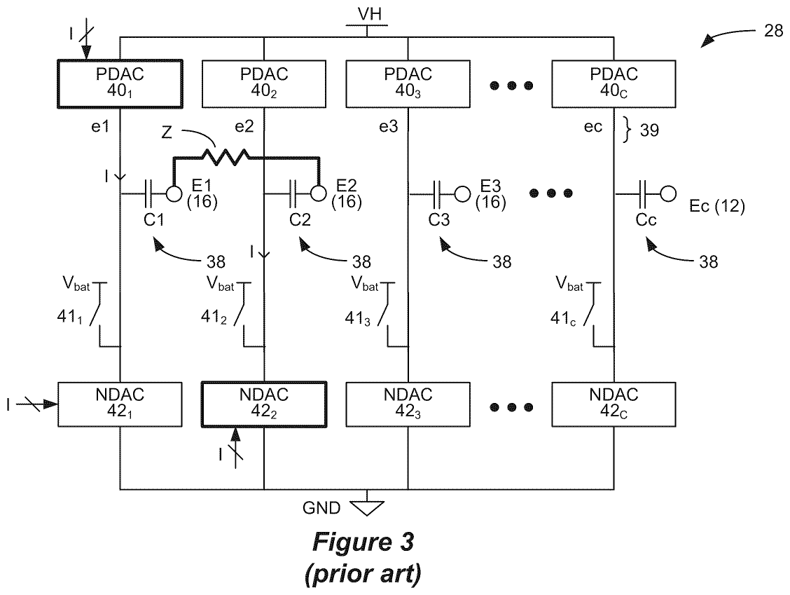

### DETAILED DESCRIPTION

A significant issue in stimulation therapy, and Spinal Cord Stimulation (SCS) therapy in particular, is determining the stimulation parameters that are best able to treat a patient's symptoms. As noted above, SCS stimulation parameters can include features like the amplitude of stimulation (I), the pulse width (PW) of the stimulation pulses, and the frequency (F) at which the stimulation pulses are issued. Other stimulation parameters used to place the stimulation at a location (L) in the electrode array 17 to best treat the patient's symptoms are also important. This location L is determined by the electrodes that are active to form the stimulation, the polarity of those active electrodes, and a percentage indicating a relative amount of the amplitude each active electrode should receive. As explained above, these parameters define the location of poles in the electrode array 17, and hence the location of the bipole 21.

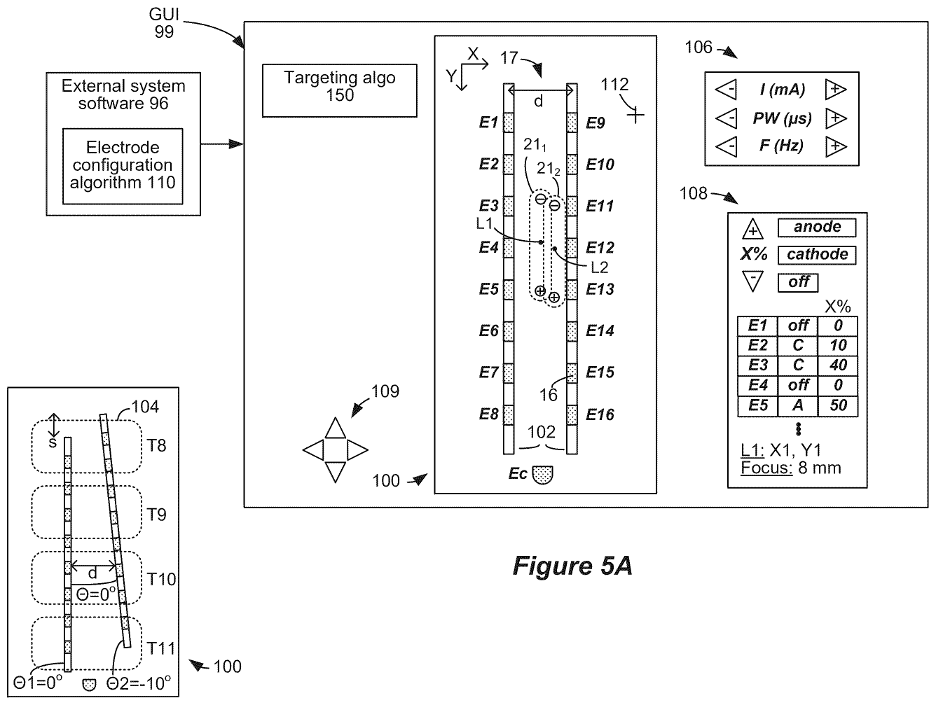

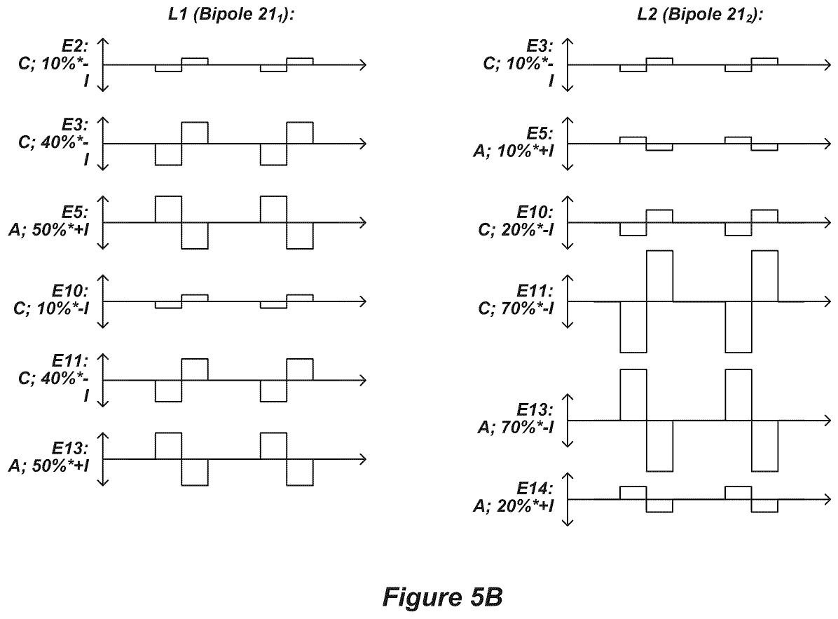

This is explained further with respect to FIGS. 5A and 5B. FIG. 5A shows GUI 99, which as noted earlier can be rendered by external system software 96 of the external system, which is in this example is assumed to be a clinician programmer 70. The GUI 99 includes a leads interface 100 which typically provides images 102 of the leads 15 (or paddle) comprising the electrode array 17.

The leads interface 100 may or may not include additional information. For example, if the relative position of the leads 15 in the patient are known, the lead images 102 may reflect such relative position, as shown in the leads interface at the bottom left in FIG. 5A. In this more complicated example, additional information regarding lead positioning is shown. For example, it can be seen that the two leads are spaced (x) in the patient's spinal canal at a particular distance d. Further the right lead is staggered upwards (y) by a particular distance s. Further, an angle between the leads is also shown, which can be reflected by understanding the angles of each of the leads in the patient (θ1=0°, θ2=−10°, thus reflecting an angle θ of 10 degrees between these leads).

Still further, the leads interface 100 can include additional information regarding where the leads in the electrode array 17 are positioned relative to anatomical features of the patient. For example, the lower-left image shows an overlay of anatomical images 104 reflecting the position of various of the patient's vertebrae, which shows that the electrodes in the array 17 are generally proximate to vertebrae T8-T11 for this particular patient. This additional information can come from various sources. For example, electrical impedance measurements can be made to discern the relative positions of the leads (e.g., d, S, and θ), as is known in the art. The additional information regarding where the leads are located relative to patient anatomical structures can also come from fluoroscopy images or other imaging techniques, which can be mapped to the location of the leads and reflected as relevant anatomical images 104 in the leads interface 100 accordingly. Such leads imaging information may be available because imaging may have occurred during the leads' surgical implantation and stored with the GUI 99.

The leads interface 100 can also provide images reflective of the prescribed stimulation. For example, a bipole 21₁ is shown, including its anode pole (+) and cathode pole (−). These poles can be formed by selecting stimulation using a parameters interface 106 and an electrode configuration interface 108. The parameters interface 106 allows parameters affecting the shape of the stimulation waveform at active electrodes to be selected, such as amplitude (I), pulse width (PW), and frequency (F). Although not shown, parameters interface 106 can include other input to define waveform shape such as whether monophasic or biphasic pulses are used, whether passive recovery will be used, whether the pulses will be bursted or duty cycled on and off, etc. The electrode configuration interface 108 provides inputs to allow the active electrodes to be selected, to define whether those electrodes are to act as anodes or cathodes, and to indicate a strength of the current those electrodes will produce. Taken together, these electrode configuration parameters define how the current In the example shown, this strength indicator comprises a percentage (X %) of the amplitude specified in the parameters interface 106. Taken together, these electrode configuration parameters define how the current will be fractionalized at the selected electrodes.

Specifying the electrode configuration in this manner allows anode and cathode poles to be formed in the electrodes array at locations that do not necessarily correspond to the physical position at any of the electrodes (what is sometimes known in the art as a virtual pole). For example, assume that the electrodes have been selected as shown to the left in FIG. 5B for bipole 21₁. Here we see that four of the of the electrodes (E2, E3, E10, and E11) have been selected as cathodes, each sharing amplitude −I with different percentages (10%, 40%, 10%, 40%, which would be affected by programming the NDACs at those electrodes accordingly). This would effectively place the cathode pole closer to electrodes E3 and E11 than to electrodes E2 and E10 (in the Y direction) while also placing this cathode pole equidistantly from these electrodes (in the X direction). Two of the electrodes (E5, E13) have been selected as anodes, each sharing amplitude −I with an equal percentage (50%, affected by programming the PDACs at those electrodes). This would effectively place the anode pole at E5 and 13 (in the Y direction) and equidistantly between the two (in the X direction). FIG. 5A shows these positions for the cathode and anode poles, which likewise establishes a midway location L1 for bipole 21₁. (X1, Y1), which may be shown in the electrode configuration interface 108. (This interface 108 could also provide the X, Y positions of the anode and cathode poles themselves). Furthermore, with the positions of the cathode and anode poles determined, a focus can be determined, i.e., a distance between these poles, which can also be reflected in interface 108.

Determining the position of the anode and cathode poles in the electrode array based on the fractionalization of the current (and hence determination of the location L1 of the resulting bipole 211) is assisted by use of an electrode configuration algorithm 110. As shown in FIG. 5A, this algorithm 110 can operate as part of the external system software 96 used to render GUI 99. Examples of the electrode configuration algorithm 110 are disclosed in U.S. Pat. No. 10,881,859, which is incorporated herein by reference in its entirety, and hence not further discussed. This algorithm 110 can operate in reverse to determine the fractionalization of the current based upon a selected location for the bipole 21₁. In this regard, GUI 99 includes inputs to allow a clinician to select a location (such as L1) for a bipole (such as 21₁). For example, a mouse cursor 112 can be used to point and click at any location in the electrode array 17 to establish L1. (Alternatively, L1 (X1, Y1) could be entered in interface 108). If the focus of the bipole 21₁ is known or specified (or has been entered at 108), the positions of the anode and cathode poles can be determined. From these anode and cathode pole positions, the electrode configuration algorithm 110 can determine the electrode fractionalization—i.e., the active electrodes, whether they act as anodes or cathodes, and their relative strengths (X %). This electrode fractionalization can then be automatically populated in interface 108 if desired.

The electrode configuration algorithm 110 can also allow a bipole to be moved in the electrode array. Suppose bipole 21₁ is moved to a new location (21₂) as shown in FIG. 5A. This can occur by having the user click on location L2 in the leads interface, or by using direction arrows 109 to gradually move the bipole (slightly down and to the right). At each step, the electrode configuration algorithm 110 can compute a new electrode fractionalization as necessary to place the bipole at the new location. FIG. 5B shows an example of this new fractionalization at electrode 21₂. For example, notice that moving the location from L1 to L2 places the cathode pole closer to E11, which results in the algorithm 110 providing a higher amount of cathodic current at that electrode (from 40%*−I to 70%*−I). To reflect the new positions of the anode and cathode poles, some electrodes are no longer used (e.g., E2), and new electrodes are used (E14), etc. In short, the GUI 99 allows the positions of stimulation poles, and hence the location L of the stimulation generally, to be defined and moved in the array, particularly (but not necessarily) as assisted through use of the electrode configuration algorithm 110. Note that the electrode configuration algorithm 110 can operate in the same manner if additional information about the relative position of the leads 15 (e.g., d, s, θ) are known. If this information is not known, the algorithm 110 can still generally operate by assuming nominal values for these parameters (d=x, S=0, θ=0) to approximately place stimulation at the correct position in the electrode array 17.

In any event, GUI 99 allows a clinician to position stimulation in the electrode array 17 in both X and Y directions, with electrode configuration algorithm 110 operable to determine the stimulation parameters (e.g., the current fractionalization) necessary to position the stimulation at a desired location. This provides good flexibility for the clinician when attempting to locate the stimulation to treat or “cover” a patient's pain to provide best therapeutic results. However, this flexibility can also make determining an optimal location for the stimulation laborious for clinician and patient, as there are many different X, Y combinations that could be potentially tested. Without guidance, the clinician may need to randomly and repeatedly apply stimulation at these different locations, and receive feedback from the patient whether the stimulation adequately covers their pain, which is inefficient.

This concern can be mitigated somewhat, because a clinician might have an informed idea where stimulation would be best located in the electrode array 17 based on a patient's symptoms, knowledge about anatomy, and other information that may be present and accessible by the GUI 99. In this regard, a clinician will generally have some understanding of how the site of a patient's pain translates to where stimulation should be logically applied in the patient's spinal column, and hence in the electrode array. For example, pain in the lower back may be affected by nerves innervating the spinal column at a particular rostro-caudal vertebral level, such as for example at vertebral level T9. Because the clinician may have some idea where the electrode array 17 has been implanted in the patient (e.g., T8-T11), such as from the anatomical image 104 information discussed above, the clinician may initially assume that providing stimulation in the electrode array 17 using electrodes proximate to this vertebral level (T9) would be expected to provide suitable therapy for the patient. Imaging information may further include information whether the leads in the electrode array 17 are staggered(s), angled (θ), and the distance between them (d), as discussed earlier, which may further assist the clinician in locating the stimulation in the electrode array 17.

Still further, the clinician will typically also have some indication of the medio-lateral (right-left) location of where the pain is occurring in the patient's body, which can also inform as to where the stimulation should be located. For example, if the patient's pain corresponds to vertebral level T9 on the right side of their body, recruitment of nerves on the right side of the spinal column at this vertebral level would be logical, thus suggesting that the stimulation located towards the right side of the electrode array 17 would provide suitable therapy (like as is shown for bipole 21₁ for instance). In short, various pieces of information may mitigate the need for the clinician to test all possible X, Y locations in the electrode array, and instead may suggest that only a smaller subset of logical locations should be tested.

Nevertheless, such guiding information may be unreliable, or may be lacking altogether. For example, while imaging information can show the relationship between the location of the electrode array 17 and a patient's anatomical structures (e.g., images 104), such imaging information may be lacking, or may not be accessible to the clinician within GUI 99. Imaging information may also not accurately reflect lead stagger (s), angle (θ), or distance (d), or these parameters may not be accurately mapped to anatomical structures in the GUI 99. In this regard, note that not all GUIs 99 account for lead stagger (s), angle (θ), or distance (d), and instead may only present the leads as shown in FIG. 5A (main) in idealized positions assuming no angle or stagger (s=0, θ=0), and with the leads at some preset distance (d=x). Even if imaging information is present in the GUI 99, leads can shift position in the spinal column over time, meaning that initially accurate imaging information may be out of date and no longer properly reflective of the position of the electrode array 17 in the patient.

Another complicating factor in determining a stimulation location in the electrode array 17 relates to the positing of the electrode array relative to the patient's physiological midline. As relevant here, this midline generally separates right and left innervating nerve branches. However, this midline is not necessary at the absolute medio-lateral center of the patient's body at the location where the electrode array 17 was implanted. Therefore, even if the electrode array 17 is positioned at the patient's medio-lateral center (e.g., with left and right leads perfectly spanning the center), the midline would not necessarily be at the medio-lateral center of the electrode array (i.e., in the X direction). This can make targeting more difficult. For example, the clinician may place the stimulation in the center of the array 17, expecting the patient to report feeling sensations, such as paresthesia, equally on both side of the body. If however, the patient's physiological midline is right of center, the patient may instead report left-affecting paresthesia. Uncertainty regarding the location of the electrode array 17 relative to the physiological midline is further exacerbated because imaging cannot detect this midline. This makes medio-lateral targeting of pain on a particular side of the patient's body difficult.

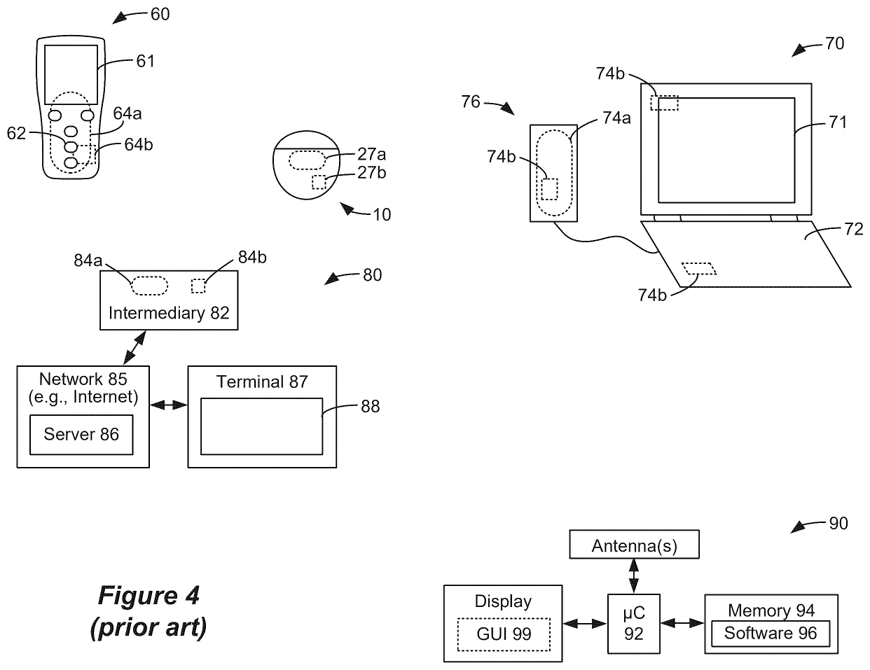

The inventors seek and disclose herein a simpler way of quickly determining an optimal location for stimulation in the electrode array 17 to best treat the patient's symptoms, which is implemented by use of a targeting algorithm 150. Targeting algorithm 150 preferably comprises part of the external system software 96 (FIG. 4) and may thus be implemented as part of the GUI 99. Thus, like the GUI 99, the targeting algorithm 150 may comprise instructions stored in a non-transitory computer-readable medium, such as memory 94 resident in an external system such as the clinician programmer 70, in the form of a disk, stick, or other memory module readable by the external system, or in an external server capable of downloading the targeting algorithm 150 to an external system (using the Internet for example).

Targeting algorithm 150 does not rely on imaging information that references the electrode array to anatomical structures in the patient (although such information is still helpful and can still be considered). This is beneficial, because as noted above such imaging information may be missing or inaccurate. Further, targeting algorithm 150 does not require information reading lead stagger (s) angle (θ) or distance (d) (although again such information can be helpful if known and referenced in the GUI 99).

In lieu of such information, targeting algorithm 150 instead provides test stimulation at different locations in the electrode array, and receives as inputs an indication about the locational effect of the test stimulation in the patient's body, i.e., which dermatomes are affected. Preferably, test stimulation is provided at extreme locations in the electrode array, such as at the top and bottom (rostro-caudally). As used herein, unless specified otherwise, “top” refers to the rostral direction or location and “bottom” refers to the caudal direction or location. Stimulation at these extremes is preferably moved medio-laterally (X) in the electrode array 17 to locations at which the patient reports feeling the stimulation at the affected dermatomes equally (symmetrically) on the left and right sides of his body. These affected top and bottom dermatomes are recorded, which allows the algorithm 150 to infer positions in the electrode array 17 corresponding to the patient's physiological midline, which is useful to setting optimal stimulation for the patient at a correct medio-lateral position (in some examples). Information indicative of the top and bottom affected dermatomes can include an affected vertebral level (V) as well as a medio-lateral parameter (ML) indicative of the medio-lateral extent of such affect. Said simply, the top and bottom affected dermatomes can each be represented by one or more top and bottom V, ML coordinates, which are recorded along with the top and bottom locations L where symmetric perception is achieved.

The targeting algorithm 150 also receives as an input information indicative of the location of the patient's pain, which may also be represented as one or more pain V, ML coordinates. A position algorithm 190 within the targeting algorithm 150 can then use the top coordinates, the bottom coordinates, the pain coordinates, and the top and bottom test stimulation locations, to interpolate a location for optimal stimulation which would appear to be effective in treating the patient's pain. Once this optimal location in the electrode array is determined, the electrode configuration algorithm 110 discussed earlier can be used to determine stimulation parameters (e.g., a current fractionalization) to position optimal stimulation at that location and tested on the patient, hopefully to good effect.

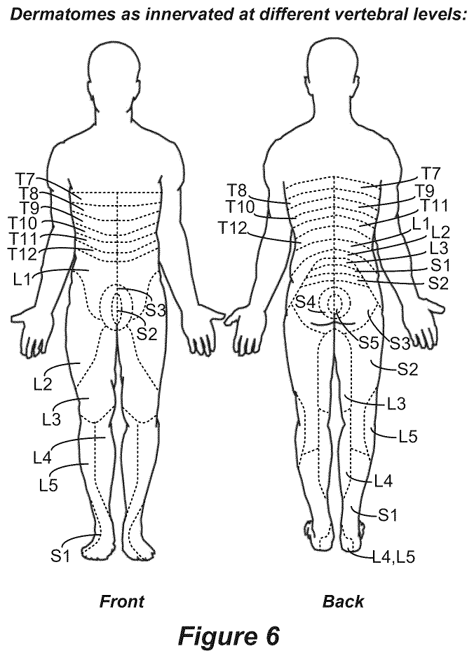

Targeting algorithm 150 as mentioned involves consideration of a patient's dermatomes, and these are shown in FIG. 6. A dermatome, generally speaking, defines an area or volume of tissue that is mainly supplied (innervated) by a particular spinal nerve. Such dermatomes may be referred to in accordance with different vertebral levels at which such spinal nerves emerge, and dermatomes ranging from vertebral levels T7-S5 are depicted. Front and back dermatomes are shown as innervated by ventral and dorsal spinal nerves, but the remainder of this disclosure focuses primarily on back dermatomes, consistent with back and related leg pain that SCS stimulation therapy typically treats. Note that these dermatomes are separated into right and left dermatomes indicative of both right and left branching nerves from the spinal column. Note that the dermatome map of FIG. 6 is merely one example of such maps, which may vary somewhat.

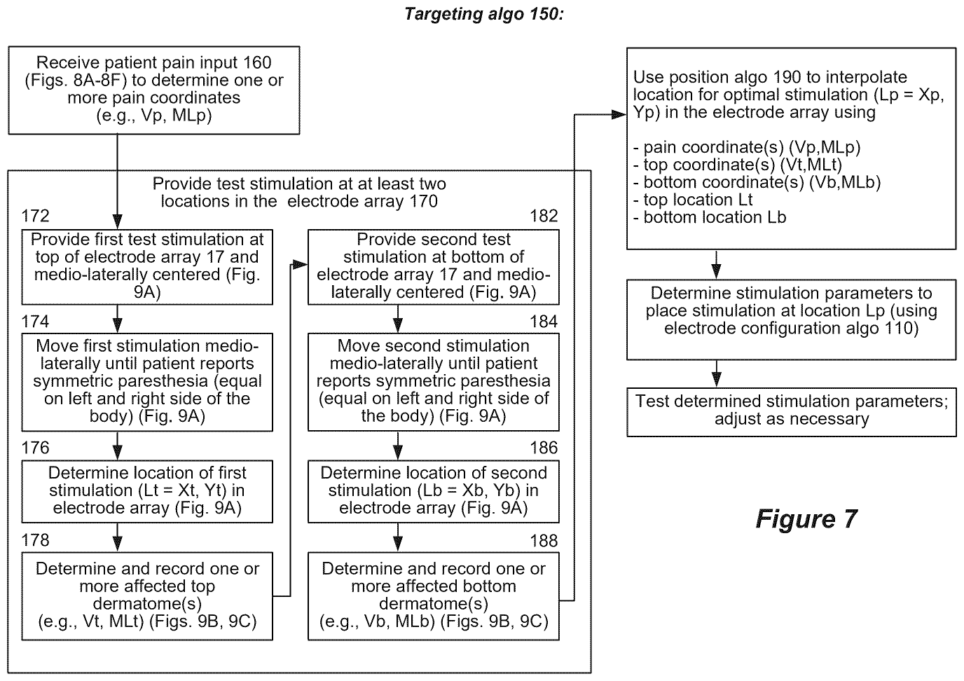

Targeting algorithm 150 is shown in flow chart form at a high level in FIG. 7, with details explained further in subsequent figures. As shown in FIG. 5A, targeting algorithm 150 can comprise a selectable option on GUI 99, which when selected can guide the clinician through various steps of the algorithm, by presenting a series of screens and options consistent with the algorithm 150 as discussed further below. One skilled in the art will understand that the steps in the target algorithm 170 as shown in FIG. 7 can occur in different orders, and that various steps could be omitted or added depending on the goals to be achieved.

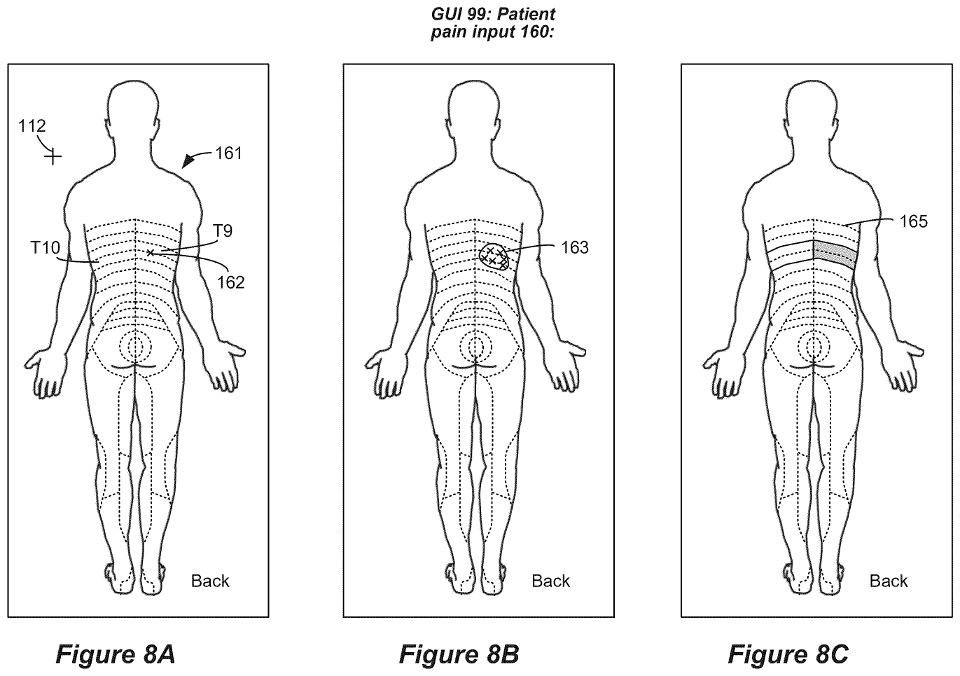

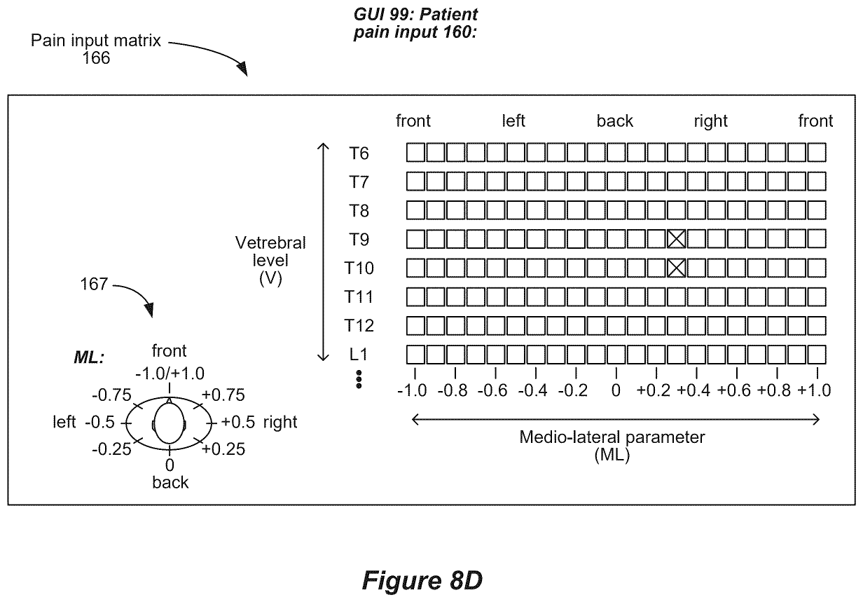

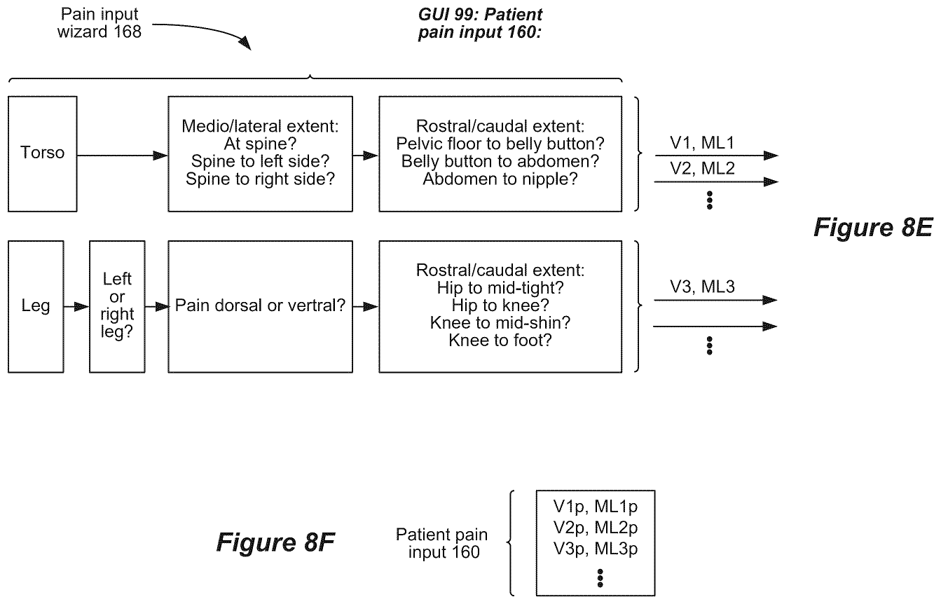

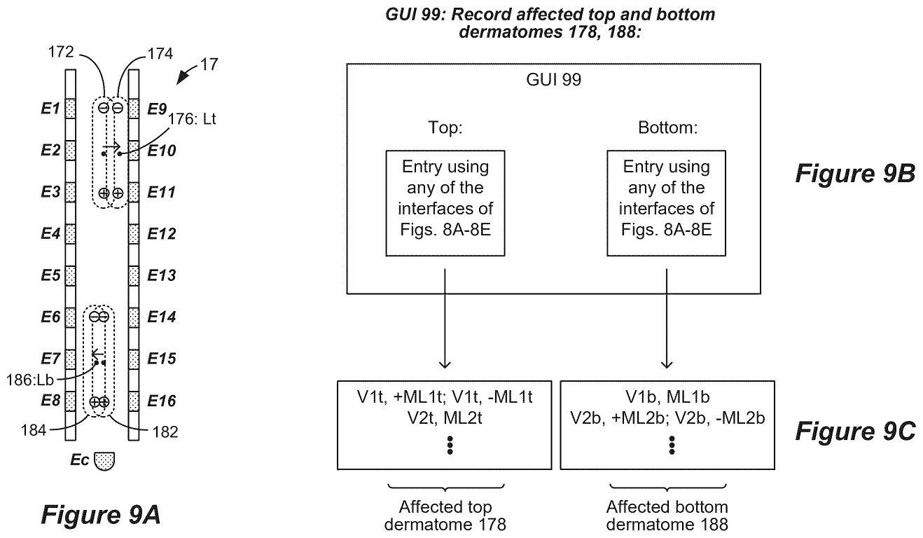

As a first step, the algorithm 150 prompts the clinician (or patient) to input information regarding where the patient is experiencing pain (when not receiving stimulation therapy). As noted earlier, such pain can occur at different vertebral levels (V) or different medio-later positions (ML), and may occur at a number of such locations. Such input can be received at the GUI 99 in a number of different ways as shown in FIG. 8A-8E. FIGS. 8A-8C allow for various forms of pain input which uses an image 161 of at least a part of a person. These images 161 show the dermatomes introduced earlier for clarity, but may not be present in an actual implementation (particularly if they would be confusing). The clinician or patient can use an input device (e.g., cursor 112) to click on the image 161 at locations indicatives of the patient's pain. In FIG. 8A, the user has selected a particular painful location 162. FIG. 8B is somewhat similar, but the user has selected several painful locations 162. The targeting algorithm can use these painful locations 162 to automatically determine a pain area 164. Alternatively, FIG. 8B may simply allow the user to draw a circle to define this pain area 164. In FIG. 8C, the body image 161 is divided up into a number of selectable regions 165. Again, for convenience, these regions 165 are shown as corresponding to the left and right dermatomes, but this could be varied, and different regions combining, sub-dividing, or overlapping various dermatomes could be defined and presented for user selection. For example, regions 165 could simply comprise a grid of selectable squares. In all of these examples, the patient pain has been indicated as occurring generally at the T9 and T10 right dermatomes.

FIG. 8D provides another means for receiving patient pain inputs in the form of a pain input matrix 166. This matrix includes a number of selectable boxes each corresponding to a different vertebral (rostro-caudal) (V) and medio-lateral positions on the patient. The vertebral levels V can correspond to the different dermatomes discussed earlier (which are in turn defined at such levels), but this is not strictly necessary and arbitrary numbers can be used indicative of rostro-caudal position of a patient's pain. The medio-lateral positions are defined by medio-lateral parameters (ML). These ML parameters and quantized in accordance with the shown graphic 167 to define various rotational positions around the patient. In this example, ML=0 indicates a position directly at the spine (back; dorsal) of the patient. Positive ML parameters define right positions on the patient, with ML=+0.5 indicating the patient's right side, ML=+1.0 indicating front (ventral), and so on. Negative ML parameters define left positions on the patient, with ML=−0.5 indicating the patient's left side, ML=−1.0 (also) indicating front, and so on. For SCS applications with pain generally occurring at the patient's back, ML parameters ranging from −0.5 to +0.5 would be most typical. Notice that pain input matrix 166 requires some knowledge of anatomy is therefore more complicated than inputs described previously (FIGS. 8A-8C), and this matrix would preferably be used by a clinician as opposed to a patient.

One or more of the boxes can be selected in the matrix 166, and consistent with earlier inputs (FIGS. 8A-8C), boxes corresponding to vertebral levels T9 and T10 have been checked for ML=+0.3, indicating pain more or less midway between the spine and their right side. In effect, this matrix 166 indicates two V, ML coordinates—9, 0.3 and 10, 0.3—which are processed by the algorithm 150 as explained further below.

FIG. 8E provides another means for receiving patient pain inputs in the form of a pain input wizard 166. This example seeks to determine one or more locations of pain through asking a series of branching questions. An initial question may ask a generally body area in which pain is felt, such as the patient's torso or leg. Assuming torso is input, next questions can inquire where on the torso such pain occurs, and may ask questions to ascertain rostro-caudal and medio-lateral extent of such pain. Medio-lateral questions may ask whether pain occurs at the spine; from the spine to the left or right side, etc. Rostro-caudal questions may ask whether pain occurs from the pelvic floor to the belly button; belly button to abdomen, etc. If the leg is selected, different questions may be asked, as shown. Although not illustrated, still further questions may be asked in response to provided answers to further define the location of the patient's pain.

Regardless of the means used to input the patient's pain, the algorithm 150 if necessary converts the patient pain inputs to the V, ML coordinates mentioned earlier with respect to FIG. 8D. FIG. 8F thus shows the outcome of use of any of the previously-described pain input methods of FIG. 8A-8E, resulting in one or more Vp, MLp coordinates indicative of location(s) of the patient's pain. (These variables are subscripted with a “p” for “pain” to distinguish them from other variables discussed next). Although not shown, one skilled will understand that the GUI 99 can include the ability to provide audio inputs (e.g., speech to text) as well.

Referring again to FIG. 7, a next step 170 provides test stimulation at at least two locations in the electrode array 17. Preferably, these locations are at extreme positions at the electrode array's borders, such as at the corners, or (as described in detail here) at the top and bottom. Testing at extreme locations is desired to understand the range of dermatomes that stimulation in the electrode array can affect. In this regard, note that the electrode array 17 is generally implanted in a patient in a manner that should span the patient's pain both rostro-caudally and medio-laterally. For example, if pain is generally occurring at T9, the electrode array 17 might span from T8-T11 in the rostro-caudal (Y) direction. Further, the electrode array 17 (leads 15 or the electrodes in a paddle) span medio-laterally (X) direction to recruit nerves on the patient's left and right sides. Testing at extreme locations, and assessing the locations of dermatomes that are affected, thus allows the algorithm 150 to interpolate a location at which optimal stimulation is indicated for the patient based on their earlier pain inputs 160, and explained further below. Although not shown, the targeting algorithm 150 and/or the clinician can determine the particular locations that are tested, which may be a function of the particular geometry of the electrode array 17 in question as well as how that electrode array has been implanted in the patient.

FIG. 7 shows an example in which testing occurs at extreme top and bottom locations in the electrode array, with the top location Lt described first in steps 172-178. At step 172, test stimulation is provided at the top of the electrode array, as shown in FIG. 9A. This stimulation, as before takes the form of a bipole, although monopolar stimulation where the electrode array carries only a single pole (with the case electrode Ec 12 being used as a current return) could also be used. Preferably, this top stimulation 172 is initially medio-laterally centered in the electrode array 17 as shown, at a location that is equidistant between the left and right leads. To affect such initial placement, the electrode configuration algorithm 110 would typically fractionalize the current equally among these left and right leads, with for example electrodes E1 and E9 sharing the cathodic current (each with 50%*−I) and electrodes E3 and E11 sharing the anodic current (each with 50%*+I). Preferably, the top stimulation is supra-perception, meaning that the patient is able to feel the effect of the stimulation as paresthesia. This can be achieved by increasing the amplitude I of the bipole 172 as necessary.

(Note: this initial placement and current fractionalization of top stimulation 172 assumes no lead stagger s or angle θ. If such non-idealities are present, the electrode configuration algorithm 110 could adjust for them as necessary to more accurately locate the stimulation in the center of the electrode array 17. However, it is not strictly required that lead stagger s, angle θ, and distance d be corrected for, or even known, when algorithm 150 is used. If such non-idealities are present and unaccounted for, the effect would be that top stimulation 172 would not in reality be initially perfectly medio-laterally centered in the electrode array 17. But this would not matter or adversely affect operation of the algorithm 150. In short, the algorithm 150 may instead simply assume that the leads aren't staggered or angled, and may otherwise conveniently ignore these non-idealities).

Thereafter, at step 174, this top stimulation is moved medio-laterally in the electrode array 17 (e.g., by the clinician using arrows 109 in FIG. 5A for example) until the patient reports feeling paresthesia symmetrically on left and right sides of his body. This is significant, because stimulation at this location corresponds to the patient's physiological midline (which as noted earlier cannot be guaranteed to be at the precise middle of the electrode array 17). Once symmetric top paresthesia has been achieved, at step 176, the algorithm 150 can determine the location Lt of the stimulation in the electrode array (Xt, Yt), using the electrode configuration algorithm 110 described earlier if necessary.

Next, at step 178, the location of dermatomes affected by this top stimulation are determined and recorded. As shown in FIG. 8B, the GUI 99 can include inputs means to receive this location information, and these input means can be the same as those used earlier when receiving the patient's pain inputs 160 (see FIGS. 8A-8E), which are not shown again for simplicity. Also similarly with the pain information, the location of dermatomes affected by the top stimulation can be recorded (or converted by the algorithm 150) to V,ML coordinate pairs indicative of the rostro-caudal and medio-lateral location of the affected top dermatome(s). Because the affected top dermatome(s) are felt symmetrically on the left and right, a given V parameter may have more than one ML value. For example, VIt is paired with both +MLt1 and −MLt1, thus indicating that stimulation is felt equally on the left and right sides at VIt and with the same medio-lateral extent (both right and left). However, it may not be possible to achieve stimulation which feels completely symmetric to the patient, and thus different MLt values may be paired with the same Vt level even when best symmetry is achieved. In any event, one or more Vt, MLt pairs for the top affected dermatomes may be recorded (with subscript “t” denoting “top”), as shown in FIG. 9C.

Steps 182-188 are analogous steps for providing test stimulation at the bottom of the electrode array 17, and recording the bottom location Lb and the affected bottom dermatomes Vb, MLb (with subscript “b” denoting “bottom”). At step 182, test stimulation is provided at the bottom of the electrode array, and initially medio-laterally centered. At step 184, this bottom stimulation is moved medio-laterally in the electrode array 17 until the patient reports feeling paresthesia symmetrically on left and right sides of his body. Once symmetric bottom paresthesia has been achieved, at step 186, the algorithm 150 can determine the location Lb of the stimulation in the electrode array (Xb, Yb), which like location Lt corresponds to the patient's physiological midline. Next, at step 188, the location of dermatomes affected by this bottom stimulation are determined and recorded as before, leading to the recording of one or more Vb, MLb pairs for the bottom affected dermatomes, as shown in FIG. 9C.

Because the electrode array 17 is implanted to generally span recruitable locations corresponding to the patient's pain, note that painful dermatomes recorded earlier (Vp) would be expected to be between the top and bottom affected dermatomes (Vt and Vb). In this regard, top and bottom test stimulation do not necessarily need to occur at “extreme” top and bottom locations of the electrode array. Stimulation provided at any two locations which span the patient's pain can provide the same information. Test stimulation may also be provided at more than two test locations. For example, test stimulation may be provided at the four extreme corners of the electrode array. This would allow the location of affected dermatomes to be determined at these four points extremes. Stimulation at these locations may not produce symmetrical paresthesia for the patient, but may nevertheless allow the location of the physiological midline to be inferred. For example, when testing at the top left corner of the electrode array 17, the user may indicate that the stimulation is felt at a particular left medio-lateral position, and/or with a particular intensity. Similar information procured from testing at the top right corner can allow the algorithm 150 to infer or interpolate the location of Lt, and the same goes for testing the bottoms corners, yielding Lb.

Referring again to FIG. 7, a position algorithm 190 is used to interpolate an optimal location Lp=Xp, Yp in the electrode array 177 at which applied stimulation will treat the patient's pain. Generally speaking, this position algorithm 190 preferably uses at least some of the information determined to this point, including: pain coordinate(s) (Vp, MLp); top coordinate(s) (Vt, MLt); bottom coordinates (Vb, MLb); and the top and bottom locations Lt and Lb corresponding to the patient's physiological midline. As will be shown, this information allows an optimal stimulation location Lp to be determined to treat pain at the correct vertebral and medio-lateral positions. While use of top and bottom coordinates are illustrated as an example, position algorithm 190 can receive and coordinates tested at any extreme location (e.g., at the corners as mentioned above), or more generally at any at least two tested locations.

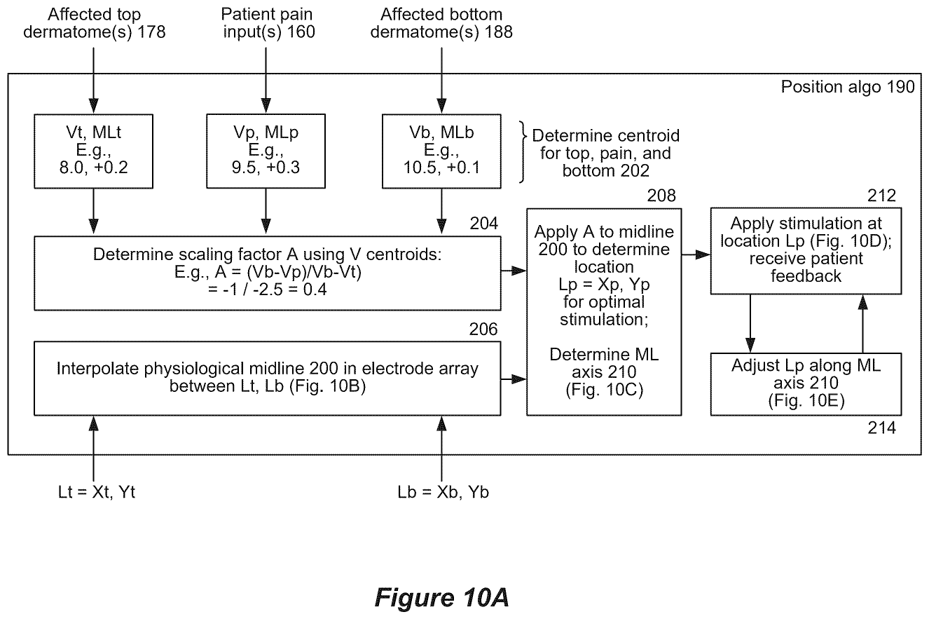

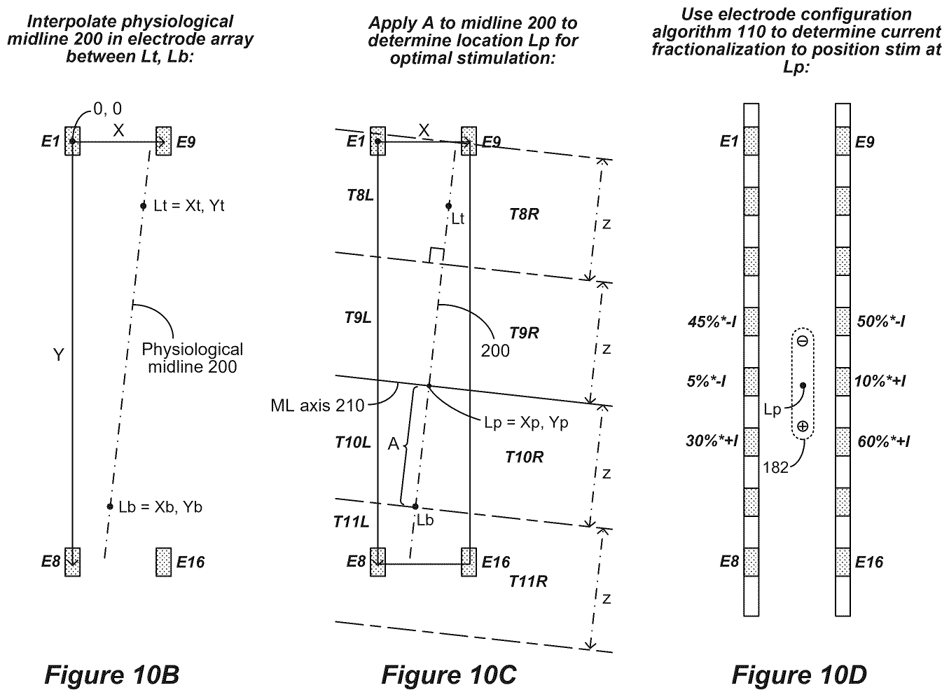

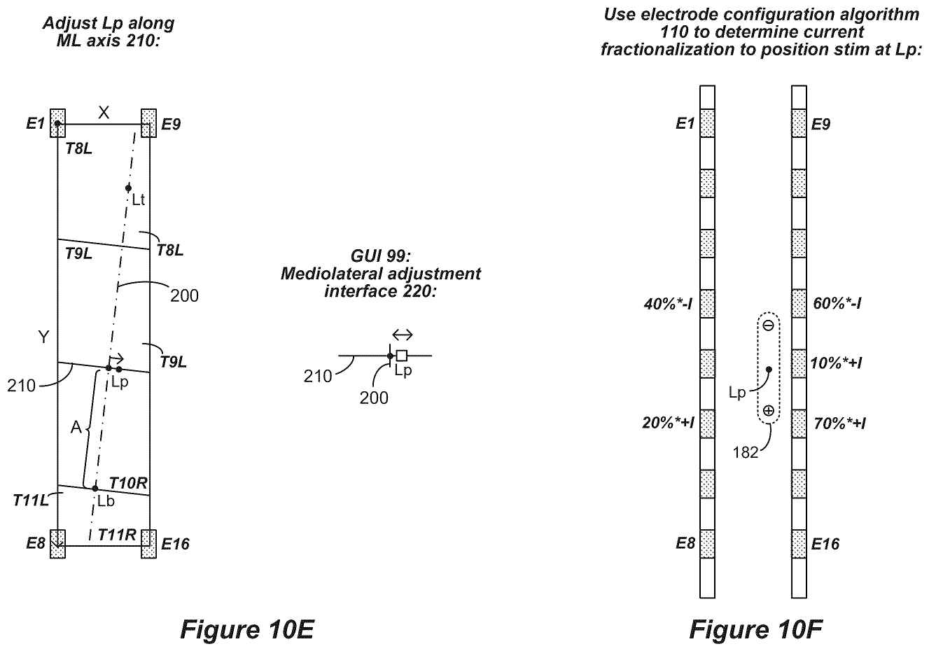

Position algorithm 190 can be implemented differently, and a first example is described with reference to FIGS. 10A-10D. First, the algorithm 190 assesses the one or more (V,ML) coordinates for the affected top dermatome(s) (178), the patient's pain (160), and the affected bottom dermatome(s) 188 to determine a single coordinate for each (202). For example purposes and to better understand the position algorithm 190, these coordinates are assumed to be Vt, MLt=8, +0.2; Vp, MLp=9.5, +0.3; and Vb, MLb=10.5, +0.1. These single coordinates can be determined in different manners, but in one example comprises some type of averaged value (e.g., a weighted average; a centroid; etc.). These single coordinates can also be weighted consistently with manners in which they might have been input.

Next, a scaling factor A is determined using the rostral-caudal variable values V, which reflects the value of Vp relative to Vt and Vb (204). This scaling factor A can be determined in different manners, but in the example shown ranges from 0 to 1 and reflects Vp's position relative to Vb. Thus, in this example, A=0.4, indicating that Vp is 40% higher than Vb (and 60% lower than Vt).

At step 206, the location of the patient's physiological midline 200 in the electrode array is interpolated using locations Lt and Lb. By way of review, these locations correspond to locations where stimulation felt symmetric to the patient on their left and right sides. The interpolated midline 200 can comprise a line between these two locations as shown in FIG. 10B. Interpolated midline 200 could also comprise a best-fit line between multiple locations if more than two were tested earlier. Although not shown, one skilled will understand that midline 200 can be expressed as a function of X and Y in the electrode array 17. Note as shown that midline 200 may be tilted within the X, Y coordinate system of the electrode array 17. This can result for a number of reasons. For example, the electrode array 17 (e.g., its leads) may not be parallel as implanted, or implanted perfectly rostro-caudally. Alternatively, the patient's physiologic midline itself may simply not be perfectly rostro-caudal at the location of the electrode array 17. While it's important to understand electrically where the midline 200 is positioned, the reason why it may deviate may not be understood and ultimately doesn't matter in the application of algorithms 150 or 190.

At step 208, scaling factor A is applied along the midline 200 to determine an optimal stimulation location Lp=Xp, Yp in the electrode array 17, as shown in FIG. 10C. Consistent with A as calculated earlier, Lp has been located along midline 200 40% above Lb and 60% below Lt. In this regard, the position algorithm 190 assumes that dermatomes are linearly scaled through the extent of the electrode array 17 consistent with Vt, Vt, and Vp. These dermatomes are overlaid in FIG. 10C (and may be shown in the GUI 99 as well), thus providing a map associating stimulation locations in the electrode array 17 and the dermatomes affected by such stimulation. Thus, notice that Lt occurs in the middle of dermatome T8, because Vt=8.0. Lb occurs at the boundary between T10 and T11, because Vb=10.5. Lp occurs at the boundary between T9 and T10, because Vp=9.5. Further, midline 200 more specifically separates the left and right dermatomes in the map (e.g., T8R, and T8L).

At step 208, it is preferable that position algorithm 190 also determine a medio-lateral (ML) axis 210 at the location of Lp. This ML axis 210 as shown is preferably perpendicular to midline 200, and thus may be tilted as well. This ML axis 210 can also be useful when determining an optimal stimulation location Lp, as it denotes more accurately the manner in which location Lp can be adjusted to affect medio-lateral adjustment of the stimulation in the patient, as explained further below. Like midline 200, ML axis 210 can be expressed as a function of X and Y in the electrode array 17.

Once Lp is determined, it can be tested by applying stimulation at this location (212). Applying stimulation at this location Lp can be assisted by use of the electrode configuration algorithm 110 discussed earlier, which can receive Lp and determine a current fractionalization at the electrodes necessary to locate the stimulation at Lp. An example of how algorithm 110 could fractionalize the current to locate stimulation at Lp is shown in FIG. 10D. Once stimulation at Lp is applied, the clinician can receive feedback from the patient as to its effectiveness in covering the treating the patient's pain. It should be noted that it may not be possible (for the electrode configuration 110) to place the stimulation exactly at point Lp as calculated by position algorithm 190, due to differences in the resolutions of the electrode configuration algorithm 110 and in the Lp computation. Nevertheless, one skilled in the art will understand that applying stimulation at Lp includes applying stimulation as close as possible to Lp given such system limitations.

Notice that Lp at this point is on the interpolated midline 200, meaning that it would be expected that the patient reports feeling symmetric paresthesia when stimulation is applied at Lp. This may be a suitable therapeutic result, even if the patient's pain as input earlier (160, FIGS. 8A-8F) was one sided (e.g., in the assumed example where MLp=+0.3, suggesting right side pain). Even if symmetric paresthesia is not strictly required to cover the patient's pain, Lp is nevertheless established by position algorithm 190 at the correct vertebral level based on the patient's pain input (Vp=9.5). If this is suitable outcome for the patient, Lp has been optimally determined to provide therapeutic stimulation, and algorithms 190 and 150 can end.

As an optional step 214, the user may decide to adjust the location of Lp along ML axis 210 to affect treatment that is more one-sided, and this is illustrated in FIG. 10E. For example, because the patient's pain was right-sided MLp=+0.3, the clinical can move Lp along ML axis 210 to the right to adjust the stimulation to affect paresthesia more to the right in the patient. To assist the clinician in making such an adjustment, position algorithm 190 may provide a mediolateral adjustment means 220 in the GUI, and FIG. 10E shows this as a slider 220. The slider may also denote midline 200 as a reference point to give the clinician some indication which positions on the slider 220 would affect left or right paresthesia for the patient. If optimal location Lp has been adjusted away from the midline 200 as is shown in FIG. 10E, the algorithm 190 can recompute the current fractionalization to properly place the stimulation in the electrode array 17 as shown in FIG. 10F.

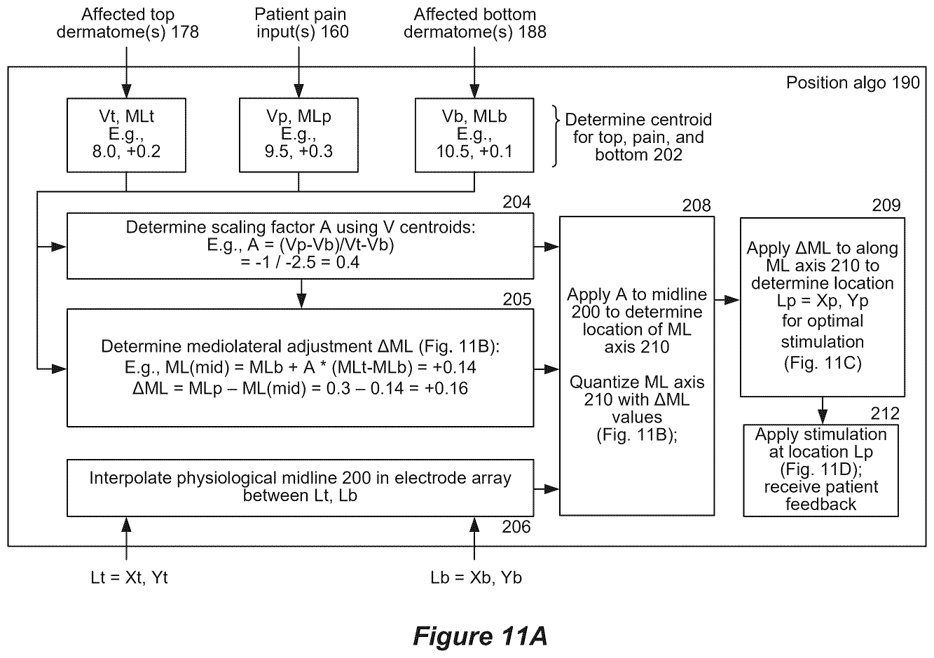

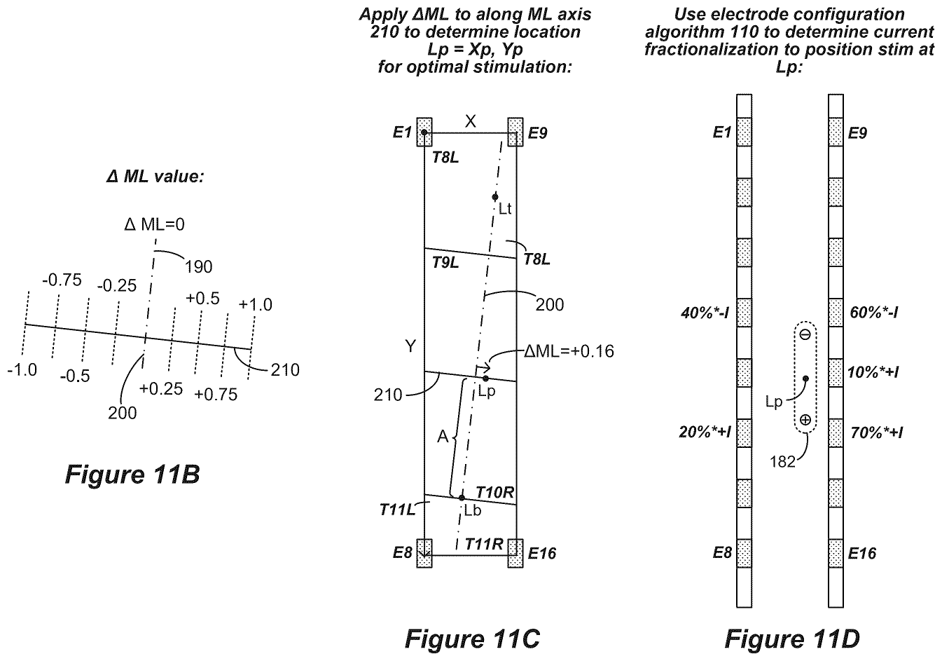

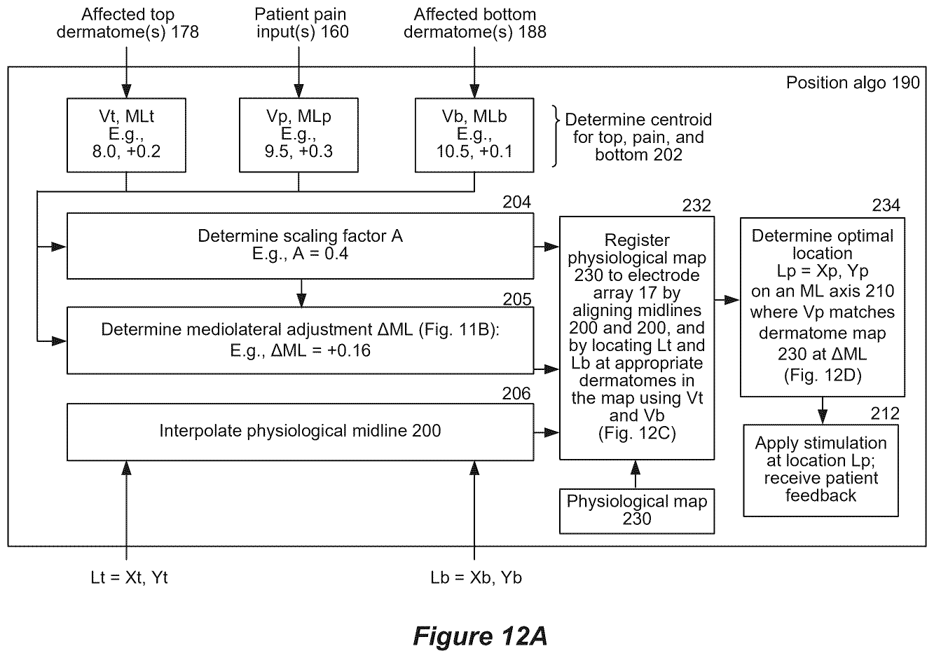

Position algorithm 190 is shown in a different embodiment in FIG. 11A-11D. In this example, the optimal location Lp is additionally automatically determined at a position at an appropriate point on the ML axis 210. As shown below, this can occur by considering the medio-lateral displacement of patient pain (MLb) and the top and bottom affected dermatomes (MLt, MLb) to automatically determine an appropriate offset (ΔML) along which Lp can be shifted away from the midline 200 along the ML axis 210. As such, this embodiment of position algorithm 190 may not require manual medio-lateral adjustment of Lp (e.g., using slider 220, FIG. 10E), although such manual adjustment can also be used.

As shown in FIG. 11A, this example of position algorithm 190 can take initial steps described earlier. For example, the algorithm 190 can once again assess the one or more (V,ML) coordinates for the affected top dermatome(s) (178), the patient's pain (160), and the affected bottom dermatome(s) 188 to determine a single coordinate (e.g., centroid) for each (202). A scaling factor A can once again be computed using the V centroids (204). Locations Lt and Lb can again be used to interpolate the physiological midline 200 (206).

As a new step (205), the ML values for the patient's pain and the top and bottom affected dermatomes (centroids MLp, MLt, MLb) can be considered to arrive at an offset, ΔML. For example, ML(mid) can comprise the expected medio-lateral effect of stimulation provided along the midline 200: e.g., ML(mid)=MLb+A*(MLt−MLb)=0.14. ΔML can then be calculated as an offset between MLp and ML(mid)=0.3−0.14=0.16. It should be noted that the example of how ΔML can be computed at step 205 is only one example, and ΔML could be calculated in other manners.

Once ΔML is calculated, step 208 can as before apply scaling factor A to determine the location of ML axis 210. Then, this axis 202 can be quantized, as shown in FIG. 11B, so that AML can be applied. Here, ΔML=0 can be defined as the location where midline 200 and ML axis 210 intersect. The ML axis 210 can then be linearly quantized with ΔML values spanning from 0 to +1.0 to a right most extent in the electrode array 17, and with ΔML values spanning from 0 to −1.0 to a left most extent.

Thereafter, in step 209, ΔML as determined earlier (205) can be applied to the now quantized ML axis 210, as shown in FIG. 11C to determine an optimal location Lp to provide stimulation for the patient. Notice that this automated process logically positions this optimal location Lp to the right of the midline 200, which makes sense given the assumed input medio-lateral variables: MLt (+0.2) and MLb (+0.1) as determined at the midline 200 both suggest that stimulation is felt somewhat to the right during testing. Providing stimulation along the ML axis 210 at the midline 200 should also provide right affecting stimulation (ML(mid)=+0.14), but the patient's pain MLp is reported to be even further right (+0.3). ΔML (+0.16) accounts for this by positioning the optimal location Lp to the right of midline 200 on ML axis 210. Again, this example of position algorithm 190 is only one example, and optimal location Lp could be rostro-caudally and medio-laterally determined in different manners. Once Lp has been determined, stimulation at this location can be provided (FIG. 11D) to assess its efficacy in treating the patient (212).

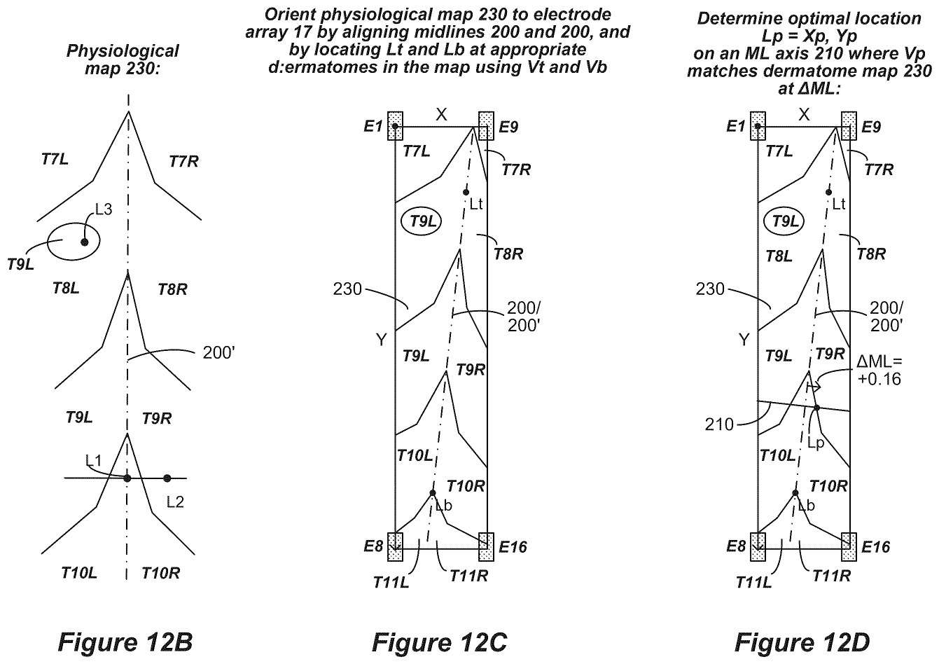

Position algorithm 190 can be modified still further as shown in FIGS. 12A-12D. This example addresses the reality that moving the position of the stimulation medio-laterally in the electrode array 17 can change the effect of where stimulation is felt rostro-caudally. As shown in FIG. 12A, shifting stimulation away from the midline 200 can start to recruit vertebral levels at lower positions, as reflected in a physiological map 230. This physiological map 230 comprises a two-dimensional map indicative of dermatomes that are recruited by stimulation at different X, Y positions in the spinal column. For example, stimulation provided at locations at or near the midline 200 (e.g., L1) may recruit nerve roots innervating T10, while moving the stimulation away from the midline (e.g., L2) will start to recruit roots innervating T9 (specifically, T9R as shown). Physiological map 230 may reflect other irregularities. For example, notice that shifting stimulation away from the midline 200 at location L3 recruits roots innervating T9 (T9L), even though T8 would be recruited at the midline 200. Physiological map 230 may be determined from experimental testing on a number of different patients, from an understanding of neurophysiology, or other sources. The data in map 230 preferably includes a midline 200′ as well as the boundaries between the different dermatomes as shown.

A physiological map 230 such as that illustrated in FIG. 12A can be stored in the clinician programmer and used within the position algorithm 190 to help determine an optimal location Lp, and this modification is shown in FIG. 12B. Steps 202-206 may be as described earlier. At new step 232, the physiological map 230 is registered to the electrode array 17, as shown in FIG. 12C. This involves aligning the midline 200 (step 206) with the midline 200′ in the physiological map. Registration can also involve fitting the map 230 so that Lt and Lb are at the correct locations. For example, because Vt=8.0, the map 230 is registered such that this point (along midlines 200/200′) falls in the middle of dermatomes T8R/L. Likewise, because Vb=10.2, the map is registered such that this point (along midlines 200/200′) falls at the boundary between T9 and T10. Note that registering the map 230 to the electrode array 17 can involve sizing or scaling the map accordingly.

At next step 234, the optimal location Lp can be determined using the Vp and ΔML values determined earlier. Specifically, step 234 can try to identify a location where the correct Vp (e.g., 9.5) value resides when stimulation is shifted by ΔML (e.g., +0.16) on an (perpendicular) ML axis 210. This is shown in FIG. 12D. Notice that at this optimal location Lp, the correct vertebral level is realized corresponding to the location of the patient pain in accordance with Vp (e.g., at the boundary of T9R and T10R), and likewise has been shifted to the right by ΔML along an ML axis 210.

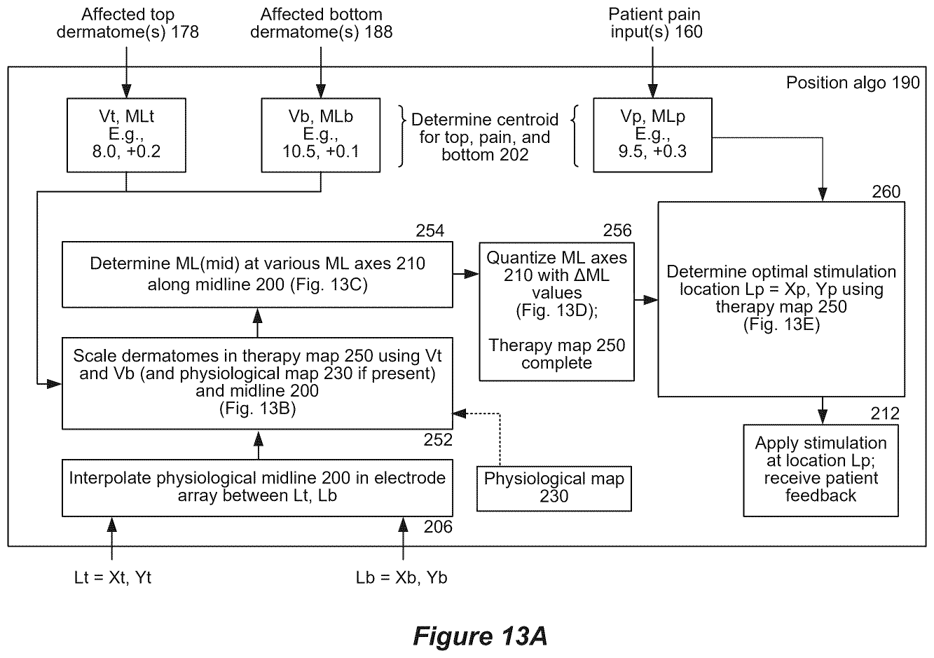

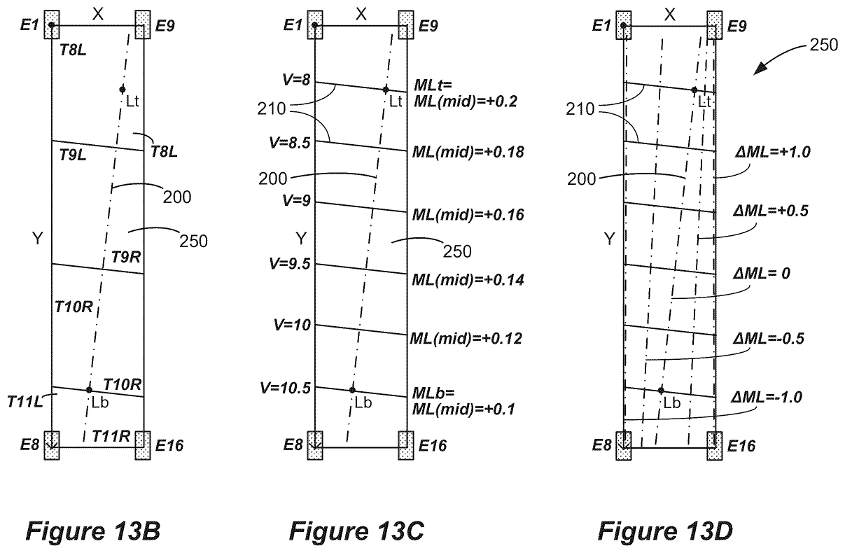

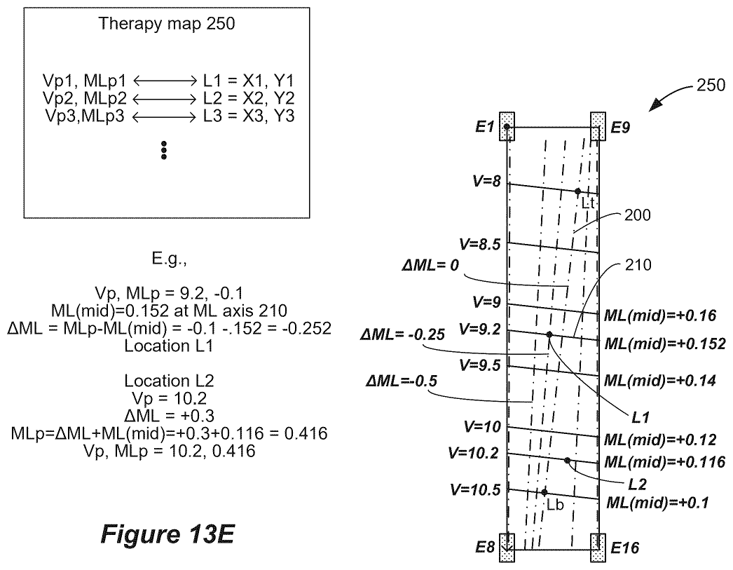

FIGS. 13A-13E show another example of position algorithm 190 operable in the targeting algorithm 150. In this example, the test stimulation (e.g., at top and bottom) is used to produce a therapy map 250 which associates locations in the electrode array 17 with one or more sites on the body affected by the stimulation at each location. This example employing the therapy map 250 is useful because it can be used to translate any patient pain input (Vp, MLp) to stimulation location Lp useful to treating that pain. This therapy map 250 is shown generically at FIG. 13E, and shows various (averaged; 202) pain input coordinates Vpi, MLi, being mapped to particular locations Li=Xi, Yi. Therapy map 250 is also useful because it can operate in reverse to determine a pain coordinate Vpi, MLi from a stimulation location Li. In this regard, the therapy map 250 is useful in its own right, and may be used in contexts outside of targeting (i.e., outside of the use of position algorithm 190 or targeting algorithm 150 more generally). For example, the therapy map 250 once determined may be displayed on the GUI 99 to give the clinician a sense of how stimulation locations will affect various dermatomes in the patient.

Details concerning how the therapy map 250 can be generated are shown in FIG. 13A in the context of the position algorithm 190, and builds upon concepts taught earlier. In this example, top and bottom test stimulation is applied and affected dermatomes 178, 188 are input and averaged as before (202). Midline 200 is also determined as before (206). At step 252, the dermatomes are scaled in the therapy map 250 using affected dermatomes Vt and Vb and the physiological midline 200. This is shown in more detail in FIG. 13B. A physiological map 230 as described earlier could also be used as part of this process, but this added complexity is not shown in FIG. 13B.

At next step 254, ML values at the midline 200 are established at various (perpendicular) ML axes 210. As shown in FIG. 13C, this can involve scaling the values MLt (e.g., +0.2) and MLb (e.g., +0.1) along the midline 200. FIG. 13C also shows the vertebral levels interpolated at each of the axes. Next, at step 256, these ML axes 210 can be quantized as before to establish AML values for each. As before, this quantization (see also FIG. 11B) can set the ΔML values such that values +1.0 and −1.0 occur at the right and left most extent of the electrode array 17, such as is shown in FIG. 13D. This completes the therapy map 250. One skilled will understand that while this map 250 is shown graphically, it can be represented by a data set in the external system.

Once therapy map 250 has been established, it can be used to determine a stimulation location in the electrode array 17 corresponding to any pain input 160, at step 260. This is shown in further details in FIG. 13E. Here it is assumed that the patient pain input received (after averaging) is Vp, MLp=9.2, −0.1. Using Vp, a relevant ML axis 210 can be located that is associated with this value along the midline 200. The therapy map 250 can further determine ML(mid) at this axis 210 (+0.152), which can be scaled or interpolated as necessary from other ML(mid) values in the map, as should be obvious. From this, a ΔML value (−0.252) to apply to this axis 210 can be determined by subtracting ML(mid) from MLp. This ΔML value can be interpolated as necessary from information in the map 250 to determine location L1 along the relevant axis 210 corresponding to pain input Vp, MLp=9.2, −0.1. Once determined, stimulation at that location can be tried on the patient (FIG. 13E, 212) as before to assess the effectiveness of stimulation therapy.

Because the therapy map 250 associates electrode array locations with affected sites on the body, it can also be used in reverse to determine a body site that should be affected by stimulation provided at any location in the electrode array 17. This is shown with reference to example location L2 in FIG. 13E. Here, L2 occurs at an axis 210 associated in the map 250 with vertebral level Vp=10.2. Further, from the map 250, it can be discerned that the ML(mid)=0.116, and that L is located at a ΔML value of approximately +0.3. from this, MLp can be determined by adding ML(mid) and ΔML (0.416). In short providing stimulation at location L2 would be expected to affect the body at Vp, MLp=10.2, 0.416.

While examples of targeting algorithm 150 seek to automatically determine an optimal location Lp of stimulation for the patient in the electrode array, it should be understood that some amount of manual adjustment of that location may be beneficial. In this regard, the clinician after trying an optimal location Lp may continue to adjust this location in the electrode array 17, such as by using arrows 109 described earlier (FIG. 5A), to see if even further beneficial locations can be achieved. However, it would be expected that beneficial locations for the patient would not deviate significantly from the optimal location Lp targeting algorithm 150 provides, and as such targeting algorithm 150 provides significant benefit in finding optimal locations for stimulation more quickly. In this regard, the optimal location Lp determined by the targeting algorithm 150 is not necessarily the absolute best possible location in the electrode array 17 at which stimulation could be applied for the patient.

As explained above, the targeting algorithm 150 receives inputs indicating vertebral levels (V) and mediolateral indices/positions (ML) where the patient feels pain and where the patient feels sensations, such as paresthesia, caused by stimulation. Aspects of this disclosure relate to user interfaces such as GUIs that facilitate the collection of the data that the algorithm can use. Examples of such interfaces have been discussed above, for example, with reference to FIGS. 8A-8E. In particular, the GUIs shown in FIGS. 8A-8C provide body maps with indications of the various dermatomes that are innervated from nerves originating from the various vertebral levels. The inventors appreciate that body maps based on dermatomes may be potentially confusing to patients and may be time consuming for the clinician to use for collecting the relevant data. Thus, aspects of the disclosure provide more intuitive interfaces for speeding the data collection.

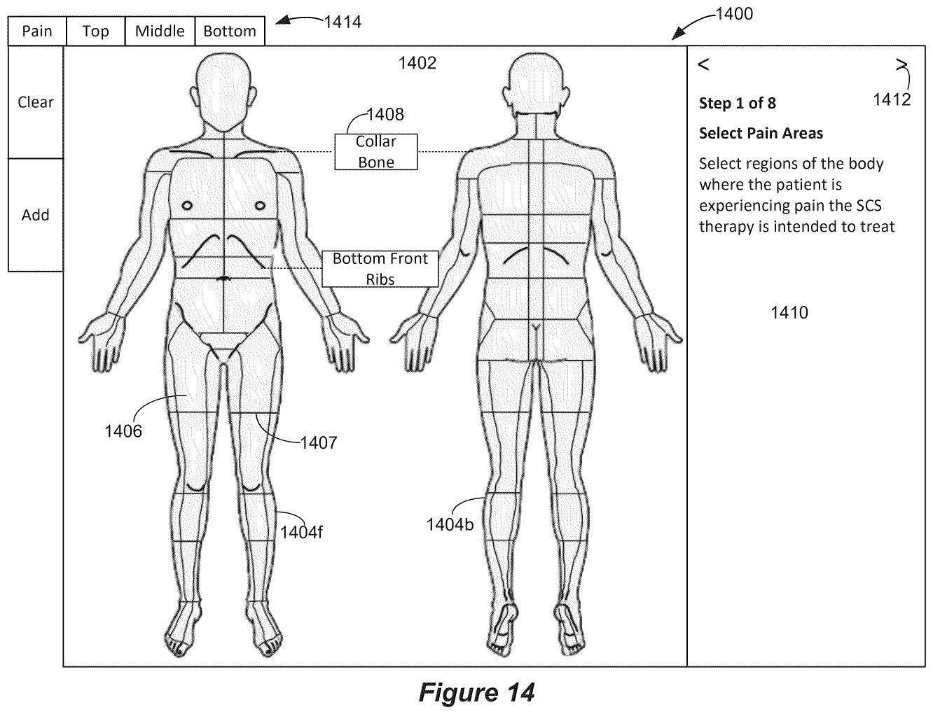

FIG. 14 illustrates an example of an embodiment of a GUI 1400. The GUI 1400 comprises a body map area 1402 that depicts body maps 1404f and 1404b, representing the front and back of the patient, respectively. The body maps 1404f and 1404 b may be divided into selectable body map regions 1406, which may correspond to intuitive body locations that a user can use to select the pain/paresthesia (or other stimulation-evoked sensation) locations for the patient. For example, the labeled body map region corresponds to the right upper thigh. Generally, the body maps may include any number of body map regions 1406, providing varying degrees of granularity for selecting the pain/paresthesia locations. For example, the body map may contain 100 or more selectable body map regions. Notice that the body map regions in the body maps 1404 f and b are essentially divorced from any indications of dermatomes covering the body map regions, that is, the body map regions do not indicate any dermatomal boundaries. In some embodiments the body map regions may be tessellated or otherwise divided into smaller, or more granulated selectable regions. According to some embodiments, the GUI may provide multiple sets of body maps having different configurations of body map regions. This may allow a clinician to select a particular set of body maps for a patient depending on the patient's indications, for example. As one example, if a patient is being treated for diabetic neuropathy, the clinician may select a body map that provides more granularity (i.e., more body map regions) in the foot region.

In the illustrated body maps the selectable body map regions 1406 are demarcated by body map region boundaries 1407 that are visible to the user. In other embodiments, the body map region boundaries may not be apparent to the user. In such embodiments, the user may select the body map region by coloring or drawing on an anatomical location and underlying logic and/or operations correlate the user's “selection” to particular body map regions, though that correlation may, or may not, be visible to the user. As used herein, the terms “select,” “selectable,” etc. refer to the ability to mark or select a location on a body map, for example, using any of the selection tools described in more detail below.

The body maps may also include anatomical landmarks 1408 to aid in the identification of the relevant body locations. Two anatomical landmarks are shown in the illustration, but the GUI 1400 typically includes more (which are omitted for clarity). In some embodiments, the identification of the anatomical landmarks may be listed, as shown in the illustration. The landmark indicators may be toggled on and off in some embodiments. In other embodiments, the labels may appear when the user hovers their cursor over the landmark, for example.

The GUI may comprise a text panel 1410 that may be configured to guide the user while collecting the relevant information. For example, the text panel 1410 may provide step-by-step instructions for executing the data collection. The instruction panel may include navigation elements 1412 for navigating through various steps of the data collection.

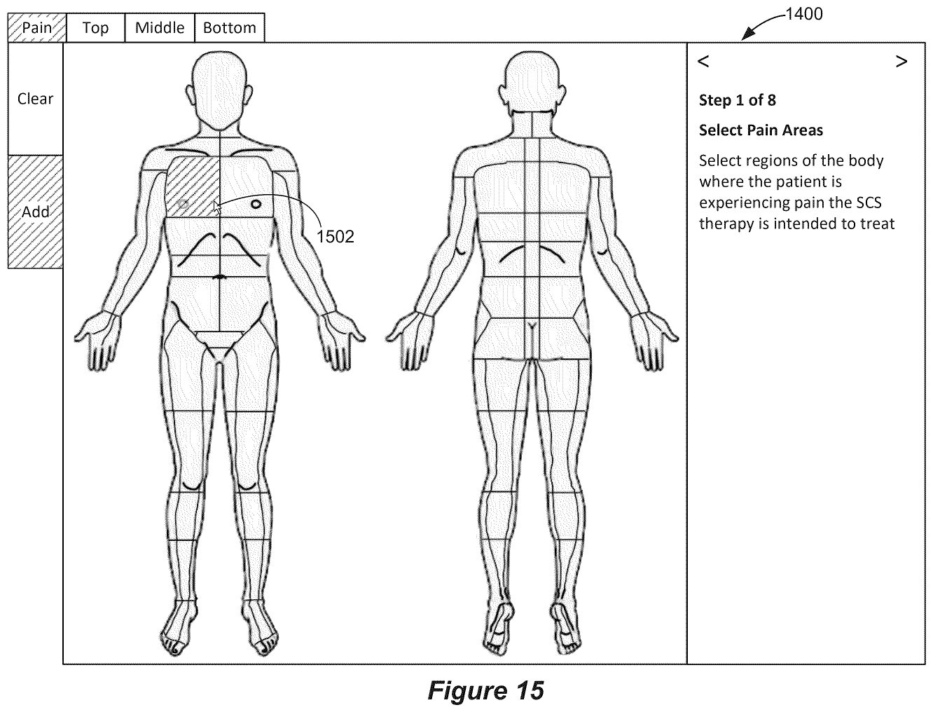

The GUI may comprise selector elements 1414 for navigating through the various functionalities of the GUI. For example, in FIG. 15 the user has selected the “Pain” and “Add” selector elements, allowing the user to use their cursor 1502 to select body map regions corresponding to body locations where the patient feels pain. The “Clear” selector element allows a user to deselect body map regions that were previously selected. The “Top, Middle, and Bottom” selector elements allow the user to indicate body map regions where the patient feels paresthesia elicited by stimulation at test stimulation points at the top, middle, and bottom of the lead array, as described above. Here it should be noted that in some embodiments, the clinician may also indicate areas where the stimulation test points result in a decrease in pain instead of, or in addition to causing paresthesia. For example, when using stimulation treatment modalities in which pain relief washes-in very quickly, the patient may be able to quickly report on body locations where they feel pain relief and those body locations may be recorded using the body maps.

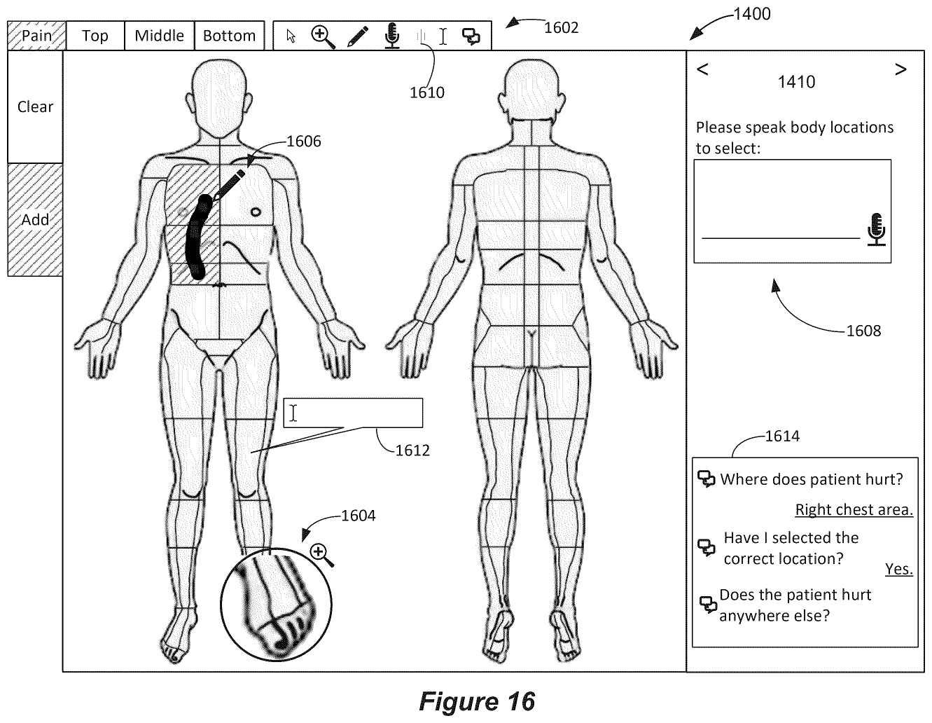

As illustrated in FIG. 16, the GUI 1400 may be configured with various tools 1602 to aid in the efficient collection of data using the body maps. For example, the GUI may be configured with a zoom or magnifying functionality or element 1604, whereby the user may magnify a portion of the body map to aid in the selection of body map regions. The magnifying element may magnify a selected portion or the whole image, and may include functionality like pinch, zoom, and pan, for example. Some embodiments may include a draw or scribble functionality represented by pen (or stylus) 1606, whereby a user may quickly scribble across multiple body map regions to select them. The GUI may be configured with a speech-to-text functionality 1608, whereby the user can speak the body map regions for selection. In some embodiments, the GUI may feature a symmetry function 1610. When the symmetry function is selected, the user may select body map regions on one side of the map and the GUI will automatically select the corresponding body map regions on the other side of the map. Some embodiments of the GUI may comprise a text/note tool 1612, whereby the user can associate notes with various body map regions. Some embodiments of the GUI may include a chat tool 1614 (e.g., a chatbot), which may prompt the user, for example through a series of questions, to type or speak the locations where pain/paresthesia is felt and to automatically select the corresponding body map regions. In some embodiments, the chat tool may be configured with natural language processing (NLP) and/or artificial intelligence capabilities to aid the data collection.

Some embodiments of the GUI may provide pre-filled or auto-selected body maps, wherein certain body map regions are preselected based on the patient's indications. For example, the clinician may preload a template body map corresponding to a patient's indication (e.g., failed back surgery syndrome, diabetic neuropathy, etc.), which has certain body map regions already selected for pain. The clinician may then deselect nonpainful body map regions or select additional body map regions, as needed. In some embodiments, the GUI and associated algorithms may be configured to store indicia of commonly selected body map regions associated with various patient indications. For example, the GUI may be configured adaptive learning algorithms to “learn” such commonly selected body map regions and to update the template to automatically fill those body map regions in future iterations or instantiations of the GUI for use with future patients.

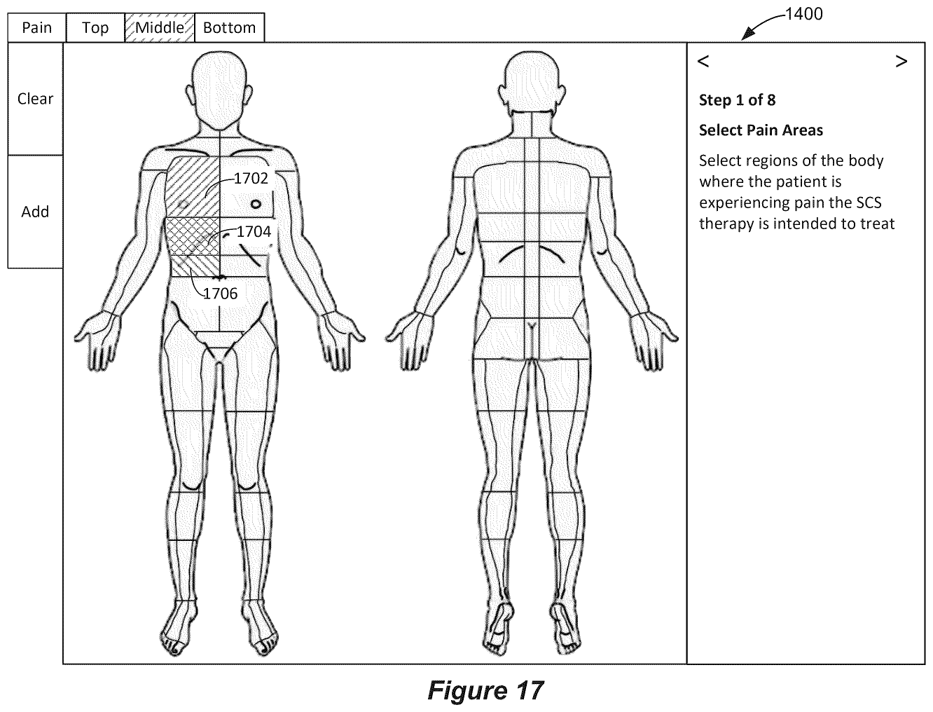

In some embodiments the body maps may be configured to color-code, or otherwise indicate, body map regions based on the patient's perception at corresponding body locations. For example, referring to FIG. 17, body map region 1702 may correspond to a body location where the patient feels paresthesia but does not feel pain; body map region 1704 may correspond to a body location where the patient feels both paresthesia and pain; and body map region 1706 may correspond to a body location where the patient feels pain, but no paresthesia.

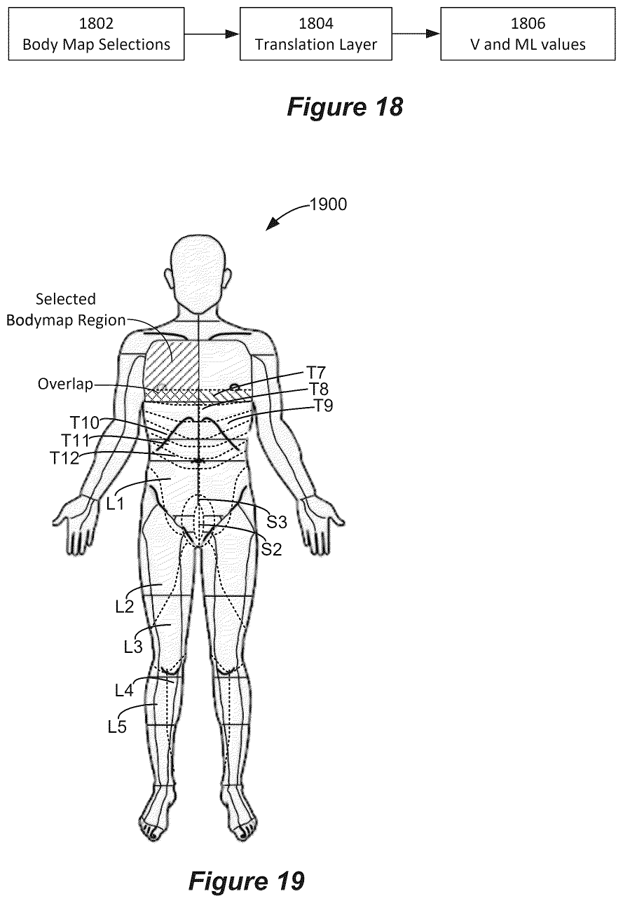

Once the body map regions correspond to the patient's pain locations and paresthesia locations (or pain relief locations) for the test stimulation locations on the lead array have been captured using the GUI's (e.g., GUI 1400) described above, those body map regions may be translated into dermatomal information used to calculate the V and ML values used by the targeting algorithm 150 described earlier. As shown in FIG. 18, embodiments of the external system software 96 may comprise a translation algorithm or translation layer 1804 for mapping the body map regions onto dermatomal locations. In some embodiments, the translation layer/algorithm is configured to evaluate and/or score an overlap of the selected body map regions with the underlying dermatomal framework.

In some embodiments, the translation layer operates by overlaying or projecting representations of body map region-based body maps, such as those illustrated in FIGS. 14-17 with dermatome-based body maps, such as those illustrated in FIG. 6. The translation algorithm determines the extent of overlap of the selected body map regions of the body map region-based maps with representations of the various underlying dermatomes. Assume that in the body map region-based body map 1900 shown in FIG. 19, the user has selected the right chest body map region as an area where pain or paresthesia is experienced. That body map region may comprise a given number of pixels or grid cells. As used herein, the pixel may refer to any type of grid cell. The body map may be projected onto a map of dermatomes, some of which are illustrated in the body map 1900, for illustrative purposes. Each of the dermatomes in the dermatome map may also comprise a given number of pixels or grid cells. Notice that the selected body map region overlaps a portion of the T7 dermatome. The extent of overlap between the selected body map region and the underlying dermatome may be used to determine if the T7 dermatome corresponds to a vertebral level associated with pain and/or paresthesia. For example, the algorithm may determine the number of pixels of the selected body map regions that overlap with pixels of the T7 dermatome.

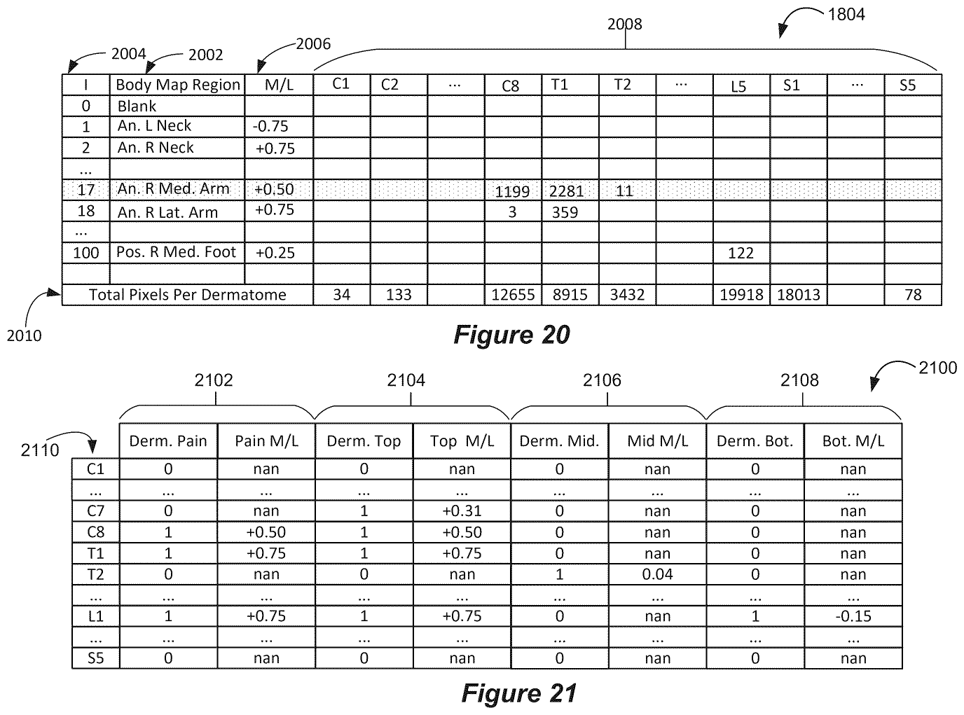

FIG. 20 shows a numeric example of a translation layer/translation algorithm 1804. The translation layer comprises an array (or bank) of all of the body map regions contained in a particular body map 2002. In the illustrated example there are 100 selectable body map regions in the body map, though there could be more or fewer, as mentioned above. Each selectable body map region is associated with an identifying index 2004 and with a mediolateral (M/L) index 2006, which may be as described above. The translation layer may also comprise an array (or bank) of dermatomes 2008. In the illustrated embodiment there are 30 dermatomes (C1-S5). Each dermatome may be represented by a given total number of pixels 2010, as shown in the illustration. For example, the number of pixels per dermatome 2010 may correspond to a number of pixels used to illustrate that dermatome in a dermatome-based body map, such as the one discussed above with reference to FIG. 19.

When a user selects a given body map region using a body map region-based body map, the translation algorithm determines the number of pixels of the selected body map region that overlaps with the pixels of a given dermatome. For example, in FIG. 20, assume that a user has selected a body map region corresponding to the anterior right medial arm from a body map. In the illustrated example, that body map region has 1199 pixels overlapping with the C8 dermatome, 2281 pixels overlapping with the T1 dermatome, and 11 pixels overlapping with the T2 dermatome.

In some embodiments, the algorithm may generate a set of basis functions using data as shown in FIG. 20. For example, there may be one basis function corresponding to each of the body map regions, wherein each basis function comprises the mediolateral index and a dermatomal coefficient indicating which (and/or to what extent) dermatomes are activated. In some embodiments, the dermatomal coefficients may simply be a one or a zero indicating whether or not the associated dermatome is activated. For example, the translation algorithm may compare the number of overlapping pixels to the total number of pixels per dermatome. In the illustrated example, the anterior right medial arm body map region overlaps with 9.5% (i.e., 1199/12655) of the pixels of the C8 dermatome. If that number exceeds a predefined ratio (e.g., 2%), then the C8 dermatome may be assigned a dermatomal coefficient of 1, meaning that it is activated. In other embodiments, the dermatomal coefficients may be other values indicative of an extent of activation.

In some embodiments, dermatomes and/or body map regions may be weighted, for example, based on importance or intensity of activation and the weights may be considered in determining the overall score for the correlation of selected body map regions to the underlying dermatomal (or other physiological) framework. For example, dermatomal locations, the stimulation of which are known to be prone to side-effects, may be weighted less, and therefore deemphasized in the overlap calculation, whereas dermatomal locations, the stimulation of which is known to likely effective may be more heavily weighted. Likewise, the patient's pain score and/or intensity of evoked sensation at a particular body map region may be used as a weighting factor for scoring the translation from body map space to dermatomal space. It is within the ability of a person of skill in the art to recognize permutations of such translation paradigms that extend beyond comparing image overlaps of the body map framework with the dermatomal framework for scoring the overlap.

Once the user has made selections from the body map region-based body map for the patient's pain locations and for the paresthesia evoked using the stimulation test points (top, bottom, middle, etc.), the translation algorithm compiles a matrix indicative of the selected dermatomes and the M/L indices of the selected body map regions. This matrix may be compiled as a mathematical (e.g., linear) combination of the basis functions described above. In this embodiment, the translation algorithm may compile a 2×30 matrix ({(M/L value)×(30 Dermatomes)}) for each of the tests (i.e., (1) pain location; (2) stimulation at top of array; (3) stimulation at bottom of array, etc.). In some embodiments, each of the 2×30 matrices may be combined into a combined matrix, such as the matrix 2100 illustrated in FIG. 21. The combined matrix 2100 comprises four 2×30 matrices 2102, 2104, 2106, and 2108, corresponding to pain location, paresthesia evoked at the top of the array, paresthesia evoked at the middle of the array, and paresthesia evoked at the bottom of the array, respectively. For each matrix, the number in the left column indicates whether the dermatome 2110 is flagged as selected (“1”) or not (“0”). The right column indicates the M/L index for the selected dermatome. For example, from the illustrated combined matrix it is apparent that the patient feels pain at the C8 dermatomal level on the right side of their body (M/L index=0.5). Thus, this matrix provides all the V and ML values that can be used by the targeting algorithm, as described above.

It should be noted that while the translation algorithms/layers described herein provide a convenient way of collecting the V and ML values to be used in the targeting algorithms described above, the translation algorithms/layers can be used for other purposes as well. Generally, the translation algorithms/layers may be used in any context when it is needed to translate from intuitive body regions to dermatomal regions. Examples may include diagnostic applications, as well as providing reports to patients and other practitioners, etc. Moreover, while the translation algorithm/layers have been described herein in the context of translating from selectable body map regions to dermatomes, the techniques can generally be used to translate from selectable regions to any underlying anatomical and/or physiological reference frame. Also, while the specific embodiments described in the disclosure primarily relate to SCS, the concepts of body maps and targeting, as described herein may also be used in other contexts, such as deep brain stimulation, peripheral nerve stimulation, etc.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An external system for programming stimulation for a spinal cord stimulator implanted in a patient to treat the patient's pain, wherein the spinal cord stimulator comprises an electrode array implantable in the patient's spinal column, the external system comprising:
a user interface and control circuitry configured to:
cause the user interface to present one or more body maps comprising selectable body map regions,
receive indications of one or more selected body map regions corresponding to body locations where the patient feels the pain,
cause stimulation circuitry of the spinal cord stimulator to provide one or more test stimulations,
receive indications of one or more selected body map regions corresponding to body locations where the patient feels sensations evoked by the one or more test stimulations, and
use a translation algorithm to determine mediolateral locations and dermatomes where the patient feels the pain and the sensations based on the selected body map regions.

2. The system of claim 1, wherein the one or more body maps do not provide any indication of dermatome boundaries.

3. The system of claim 1, wherein the translation algorithm is configured to determine overlaps of the selected body map regions with dermatomes in the patient's body.

4. The system of claim 1, wherein:
the translation algorithm is configured to generate one or more basis functions for each of the selected body map regions,
wherein each of the basis functions comprises a mediolateral index and one or more coefficients indicative of dermatomes that overlap each of the respective selected body map regions,
wherein the one or more coefficients are determined based on a number of pixels representing the selected body map regions that overlap with pixels representing respective dermatomes, and
wherein the mediolateral locations and dermatomes where the patient feels the pain and the sensations are each determined based on mathematical combinations of their respective basis functions.

5. The system of claim 1, wherein the control circuitry is configured to:
use the mediolateral locations and dermatomes where the patient feels the pain and the sensations to automatically determine one or more therapeutic locations in the electrode array where stimulation is likely to treat the patient's pain, and
provide stimulation at the one or more therapeutic locations.

6. The system of claim 5, wherein the one or more test stimulations comprise at least a first test stimulation using a first test stimulation location in the array and a second test stimulation using a second test stimulation location in the array.

7. The system of claim 6, wherein the control circuitry is configured to use the mediolateral locations and dermatomes where the patient feels the pain and the sensations to determine:
one or more pain inputs indicative of mediolateral and rostro-caudal locations on the patient's body where the patient feels the pain,
one or more first inputs indicative of mediolateral and rostro-caudal locations on the patient's body where the patient feels sensations evoked by the first test stimulation, and
one or more second inputs indicative of mediolateral and rostro-caudal locations on the patient's body where the patient feels sensations evoked by the second test stimulation.

8. The system of claim 7, wherein the control circuitry is configured to:
average the one or more pain inputs,
average the one or more first inputs,
average the one or more second inputs, and
automatically determine the one or more therapeutic locations using the averaged pain inputs, the averaged first inputs, the averaged second inputs, the first test stimulation location, and the second stimulation location.

9. The system of claim 5, wherein determining the one or more therapeutic locations in the electrode array comprises determining a midline in the electrode array corresponding to a physiological midline of the patient.

10. The system of claim 9, wherein the one or more therapeutic locations are automatically determined as a location on the midline or as an offset from the midline is determined along an axis perpendicular to the midline.

11. The system of claim 6, wherein the first and second test stimulations are moved in the electrode array using the user interface to a first test stimulation location where the sensations evoked by the first test stimulation is felt at symmetric body locations, and wherein sensations evoked by the second test stimulation is felt at symmetric body locations.

12. The system of claim 11, wherein the first and second test stimulations are moved medio-laterally in the electrode array using the user interface to first and second test stimulation locations, wherein the first test stimulation once moved is felt medio-laterally symmetrically on the body at the one or more first sites, and wherein the second test stimulation once moved is felt medio-laterally symmetrically on the body at the one or more second sites.

13. The system of claim 1, wherein the user interface comprises one or more selection elements configured to assist a user in selecting the selectable body map regions.

14. The system of claim 13, wherein the one or more selection elements comprise a cursor.

15. The system of claim 13, wherein the one or more selection elements comprise a pen or stylus, whereby a user can mark across multiple selectable body map regions to simultaneously select each of them.

16. The system of claim 13, wherein the one or more selection elements comprise a symmetry function, configured such that when a user selects a first body map region on a first half of the body map a second body map region symmetrically positioned on a second half of the body map is also selected.

17. The system of claim 13, wherein the one or more selection elements comprise a magnifying element, which when positioned over a portion of the body map magnifies selectable body map regions within the body map.

18. The system of claim 13, wherein the one or more selection elements comprise a chat tool configured to present instructions to a user to aid the user's selection of body map regions.

19. The system of claim 1, wherein the control circuitry is configured to adjust the number of selectable body map regions contained in the body maps.

20. The system of claim 1, wherein the control circuitry is configured to adjust coarseness of the selectable body map regions contained in the body maps.
